(19) United States
(12) Patent Application Publication
(10) Pub. No.: US 2026/0265335 A1
(43) Pub. Date: Sep. 10, 2026

(54) DNA-BINDING DOMAIN TRANSACTIVATORS AND USES THEREOF

(71) Applicant: University of Massachusetts, Westborough, MA (US)

(72) Inventors: Miguel Sena Esteves, Worcester, MA (US); Scot A. Wolfe, Worcester, MA (US)

(73) Assignee: University of Massachusetts, Westborough, MA (US)

(21) Appl. No.: 19/551,692

(22) Filed: Feb. 27, 2026

Related U.S. Application Data
(62) Division of application No. 17/433,269, filed on Feb. 24, 2020, now Pat. No. 12,583,902.
(60) Provisional application No. 62/810,005, filed on Feb. 25, 2019.

(51) Int. Cl. C07K 14/705; A61K 38/00; C12N 9/22; C12N 15/11; C12N 15/86

(52) U.S. Cl. CPC C07K 14/705; C12N 9/22; C12N 15/11; C12N 15/86; A61K 38/00; C07K 2319/71; C07K 2319/80; C07K 2319/81; C12N 2310/20; C12N 2750/14143

(57) ABSTRACT

In some aspects, the disclosure relates to recombinant adeno-associated viruses (rAAVs) comprising a nucleic acid encoding a fusion protein comprising a DNA-binding domain and a transcriptional regulator domain and methods of using the same. In some embodiments, expression of the fusion protein results in modified expression of a target gene in a cell.

### RELATED APPLICATION

This application is a divisional of U.S. application Ser. No. 17/433,269, filed Aug. 24, 2021, now U.S. Pat. No. 12,583,902, which is a national stage filing under 35 U.S.C. § 371 of international application PCT/US2020/019546, filed Feb. 24, 2020, which claims priority under 35 U.S.C. § 119(e) to U.S. provisional patent application Ser. No. 62/810,005, filed Feb. 25, 2019, the entire contents of each of which are incorporated by reference herein.

### REFERENCE TO AN ELECTRONIC SEQUENCE LISTING

The contents of the electronic sequence listing (U012070106US02-SEQ-KZM.xml; Size: 181,414 bytes; and Date of Creation: Feb. 23, 2026) are herein incorporated by reference in their entirety.

### BACKGROUND

The regulation of target gene expression has emerged as a major area of biomedical research. Upregulation of gene expression can correct haploinsufficient conditions resulting from decreased gene expression. Haploinsufficiency typically results when one or more loss of function mutations are present in at least one copy of a gene. AAV-based approaches of gene augmentation for treatment of diseases associated with haploinsufficiency are hampered by the packaging capacity of traditional rAAV vectors.

### SUMMARY

Aspects of the disclosure relate to isolated nucleic acids and recombinant AAV vectors for gene delivery. The disclosure is based, in part, on compositions (e.g., rAAV vectors and rAAVs) and methods for regulating the expression of target genes, wherein the target gene is haploinsufficient, such as SCN1A. In some embodiments, the disclosure provides fusion proteins comprising a DNA binding domain, such as a Cys2-His2 Zinc Finger protein (ZFP), and a transcriptional regulator domain. In some embodiments, compositions described by the disclosure comprise a fusion protein comprising a DNA binding domain (e.g., a ZFP, a Transcriptional activator-like effector (TALE) domain, etc.) fused to a transcriptional regulator domain. In some embodiments, fusion proteins described by the disclosure increase the expression of a target gene (e.g., SCN1A), and are therefore useful for treating diseases characterized by deficient expression of the target gene (e.g., diseases associated with haploinsufficiency of a target gene) in a cell or subject as compared to a normal cell or subject.

Accordingly, in some aspects, the disclosure provides an isolated nucleic acid comprising a transgene configured to express at least one DNA binding domain fused to at least one transcriptional regulator domain, wherein the DNA binding domain binds to a target gene or a regulatory region (e.g., an enhancer sequence, a promoter sequence, a repressor sequence, etc.) of a target gene (e.g. in a subject or a cell), wherein the target gene encodes a voltage-gated sodium channel (e.g., Nav1.1). In some embodiments, a target gene is a SCN1A gene. In some embodiments, a transgene is flanked by adeno-associated virus (AAV) inverted terminal repeats (ITRs). In some embodiments, the at least one DNA binding domain binds to a target gene (e.g., in a subject or a cell) and the transcriptional regulator domain modifies, e.g., upregulates, the expression of a target gene.

In some aspects, the disclosure provides a recombinant AAV (rAAV) comprising: a nucleic acid comprising a transgene encoding at least one DNA binding domain fused to at least one transcriptional regulator domain, wherein the DNA binding domain binds to a target gene or a regulatory region of a target gene (e.g. in a subject or a cell), wherein the target gene encodes a voltage-gated sodium channel (e.g., Nav1.1) and at least one capsid protein. In some embodiments, a target gene is a SCN1A gene. In some embodiments, a transgene is flanked by AAV inverted terminal repeats (ITRs).

In some embodiments, at least one DNA binding domain binds to a target gene (e.g., in a subject or a cell) and the transcriptional regulator domain modifies, e.g., upregulates, the expression of a target gene in the subject.

In some embodiments, at least one DNA binding domain binds to an untranslated region of a target gene. In some embodiments, a DNA binding domain binds to a regulatory region of the target gene, optionally an enhancer sequence, a promoter sequence, and/or a repressor sequence.

In some embodiments, a DNA binding domain binds between 2 and 2000 bp upstream or 2 and 2000 bp upstream or downstream of a regulatory region (e.g., an enhancer sequence, a promoter sequence, a repressor sequence, etc.) of a target gene.

In some embodiments, at least one DNA binding domain encodes a zinc finger protein (ZFP), a transcription-activator like effector (TALE), a dCas protein (e.g., dCas9 or dCas12a), and/or a homeodomain. In some embodiments, at least one DNA binding domain binds to a nucleic acid sequence set forth in any one of SEQ ID NOs: 5-7. In some embodiments, the at least one DNA binding domain is a zinc finger protein comprising a recognition helix encoded by a nucleic acid having a sequence set forth in any one of SEQ ID NOs: 11-16, 23-28, or 35-40. In some embodiments, at least one DNA binding domain is a zinc finger protein comprising an amino acid sequence set forth in any one of SEQ ID NOs: 17-22, 29-34, or 41-46.

In some embodiments, the at least one DNA binding domain is a zinc finger protein comprising a recognition helix encoded by a nucleic acid comprising SEQ ID NO: 11, a recognition helix encoded by a nucleic acid comprising SEQ ID NO: 12, a recognition helix encoded by a nucleic acid comprising SEQ ID NO: 13, a recognition helix encoded by a nucleic acid comprising SEQ ID NO: 14, a recognition helix encoded by a nucleic acid comprising SEQ ID NO: 15, and/or a recognition helix encoded by a nucleic acid comprising SEQ ID NO: 16. In some embodiments, the at least one DNA binding domain is a zinc finger protein comprising an amino acid sequence of SEQ ID NO: 57. In some embodiments, a ZFP that binds to a SCN1A gene comprises at least 60%, at least 65%, at least 70%, at least 75%, at least 80%, at least 85%, at least 90%, at least 95%, at least 97%, or at least 99% sequence identity to the amino acid sequence of SEQ ID NO: 57.

In some embodiments, the at least one DNA binding domain is a zinc finger protein comprising a recognition helix encoded by a nucleic acid comprising SEQ ID NO: 23, a recognition helix encoded by a nucleic acid comprising SEQ ID NO: 24, a recognition helix encoded by a nucleic acid comprising SEQ ID NO: 25, a recognition helix encoded by a nucleic acid comprising SEQ ID NO: 26, a recognition helix encoded by a nucleic acid comprising SEQ ID NO: 27, and/or a recognition helix encoded by a nucleic acid comprising SEQ ID NO: 28. In some embodiments, the at least one DNA binding domain is a zinc finger protein comprising an amino acid sequence of SEQ ID NO: 59. In some embodiments, a ZFP that binds to a SCN1A gene comprises at least 60%, at least 65%, at least 70%, at least 75%, at least 80%, at least 85%, at least 90%, at least 95%, at least 97%, or at least 99% sequence identity to the amino acid sequence of SEQ ID NO: 59.

In some embodiments, the at least one DNA binding domain is a zinc finger protein comprising a recognition helix encoded by a nucleic acid comprising SEQ ID NO: 35, a recognition helix encoded by a nucleic acid comprising SEQ ID NO: 36, a recognition helix encoded by a nucleic acid comprising SEQ ID NO: 37, a recognition helix encoded by a nucleic acid comprising SEQ ID NO: 38, a recognition helix encoded by a nucleic acid comprising SEQ ID NO: 39, and/or a recognition helix encoded by a nucleic acid comprising SEQ ID NO: 40. In some embodiments, the at least one DNA binding domain is a zinc finger protein comprising an amino acid sequence of SEQ ID NO: 61. In some embodiments, a ZFP that binds to a SCN1A gene comprises at least 60%, at least 65%, at least 70%, at least 75%, at least 80%, at least 85%, at least 90%, at least 95%, at least 97%, or at least 99% sequence identity to the amino acid sequence of SEQ ID NO: 61.

In some embodiments, the at least one DNA binding domain is a zinc finger protein comprising a recognition helix comprising the amino acid sequence of SEQ ID NO: 17, a recognition helix comprising the amino acid sequence of SEQ ID NO: 18, a recognition helix comprising the amino acid sequence of SEQ ID NO: 19, a recognition helix comprising the amino acid sequence of SEQ ID NO: 20, a recognition helix comprising the amino acid sequence of SEQ ID NO: 21, and/or a recognition helix comprising the amino acid sequence of SEQ ID NO: 22.

In some embodiments, the at least one DNA binding domain is a zinc finger protein comprising a recognition helix comprising SEQ ID NO: 29, a recognition helix comprising SEQ ID NO: 30, a recognition helix comprising SEQ ID NO: 31, a recognition helix comprising SEQ ID NO: 32, a recognition helix comprising SEQ ID NO: 33, and/or a recognition helix comprising SEQ ID NO: 34.

In some embodiments, the at least one DNA binding domain is a zinc finger protein comprising a recognition helix comprising SEQ ID NO: 41, a recognition helix comprising SEQ ID NO: 42, a recognition helix comprising SEQ ID NO: 43, a recognition helix comprising SEQ ID NO: 44, a recognition helix comprising SEQ ID NO: 45, and/or a recognition helix comprising SEQ ID NO: 46.

In some embodiments, the at least one DNA binding domain is a catalytically inactive CRISPR associated protein (Cas protein). In some embodiments, a catalytically inactive Cas protein (or “dead Cas protein”) is a dCas9 or dCas12 protein. In some embodiments, a nucleic acid or rAAV further comprises at least one guide nucleic acid (e.g., guide RNA, or gRNA). In some embodiments, the guide nucleic acid comprises a spacer sequence that targets SCN1A. In some embodiments, the guide nucleic acid comprises a spacer sequence having a nucleotide sequence of any one of SEQ ID NO: 85, 86, 89, 90, 93, or 94. In some embodiments, the guide nucleic acid comprises a nucleotide sequence of any one of SEQ ID NO: 83-94. In some embodiments, the guide nucleic acid is encoded by the nucleic acid sequence set forth in any one of SEQ ID NO: 83-94.

In some embodiments, at least one transcriptional regulator domain is a transactivator domain comprising a VP16 domain, VP64 domain, Rta domain, p65 domain, Hsf1 domain, or any combination thereof, such as a VPR domain (VP64+p65+Rta1 domains). In some embodiments, at least one transcriptional regulator domain is encoded by a nucleic acid sequence as set forth in SEQ ID NO: 47. In some embodiments, at least one transactivation domain comprises the amino acid sequence set forth in SEQ ID NO: 48.

In some embodiments, the ITRs which flank the transgene comprise an ITR selected from the group consisting of: AAV1 ITR, AAV2 ITR, AAV3 ITR, AAV4 ITR, AAV5 ITR, AAV6 ITR, AAV8 ITR, AAVrh8 ITR, AAV9 ITR, AAV10 ITR, or AAVrh10 ITR. In some embodiments, the ITR is a ΔTR or an mTR.

In some embodiments, a transgene of an isolated nucleic acid is operably linked to a promoter. In some embodiments, a promoter is a tissue-specific promoter. In some embodiments, a tissue-specific promoter is a neuronal promoter, such as SST, NYP Phosphate-activated glutaminase (PAG), Vesicular glutamate transporter-1 (VGLUT1), Glutamic acid decarboxylase 65 and 57 (GAD65, GAD67), Synapsin I, a-CamKII, Dock10, Prox1, Parvalbumin (PV), Somatostatin (SST), Cholecystokinin (CCK), Calretinin (CR), or Neuropeptide Y (NPY).

In some embodiments, a DNA binding domain of a transgene is fused to a transcriptional regulator domain by a linker domain. In some embodiments, a linker domain is a flexible linker, for example a glycine-rich linker or a glycine-serine linker, or a cleavable linker, such as a photocleavable linker or enzyme (e.g., protease) cleavable linker.

In some embodiments, an isolated nucleic acid comprises a transgene which encodes multiple DNA binding domains, for example, 1, 2, 3, 4, 5, 6, 7, 8, 9, or 10 DNA binding domains. In some embodiments, an isolated nucleic acid comprises a transgene which encodes multiple transcriptional regulator domains, for example 1, 2, 3, 4, 5, 6, 7, 8, 9, or 10 transcriptional regulator domains.

In some embodiments, an isolated nucleic acid or an rAAV is expressed in a cell or subject characterized by aberrant expression or haploinsufficiency (e.g., increased expression, or decreased expression) of a target gene with respect to a normal cell or subject. In some embodiments, an isolated nucleic acid or rAAV is expressed in a cell or subject characterized by deficient (e.g., decreased) expression of a target gene with respect to a normal cell or subject. In some embodiments, a target gene of the isolated nucleic acid or rAAV is SCN1A.

In some embodiments, an AAV capsid serotype is selected from the group consisting of AAV1, AAV2, AAV3, AAV4, AAV5, AAV6, AAV7, AAV8, AAVrh8, AAV9, AAV10, AAVrh10, or AAV.PHPB.

In some aspects, the disclosure provides methods of regulating expression of a target gene. In some embodiments, methods of the disclosure comprise administering an isolated nucleic or rAAV as described herein to a cell or subject that expresses a target gene, wherein the subject is haploinsufficient for the target gene (e.g., haploinsufficient for SCN1A). For example, in some embodiments, expression of a target gene, such as SCN1A, in the cell or subject is deficient (e.g., decreased) with respect to target gene expression in a normal cell or subject. In some embodiments, a cell to which an isolated nucleic acid or rAAV is administered is a neuron. In some embodiments, a neuron is a GABAergic neuron.

In some embodiments, administration of an isolated nucleic acid or rAAV results in target gene expression (e.g., SCN1A expression) that is increased by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to a subject that has not been administered an isolated nucleic acid or rAAV. In some embodiments, administration of an isolated nucleic acid or rAAV results in target gene expression (e.g., SCN1A expression) that is increased by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to target gene (e.g., SCN1A) expression in the subject prior to being administered the isolated nucleic acid or rAAV.

In some aspects, this disclosure provides a method of regulating gene expression (e.g., expression of SCN1A) in a subject, wherein an isolated nucleic acid or rAAV as described herein is administered to a subject that expresses a target gene. In some embodiments, expression of the target gene in a subject is aberrant (e.g., increased or decreased) with respect to a healthy subject. In some embodiments, a subject is or is suspected of being haploinsufficient with respect to expression of a target gene relative to a healthy subject.

In some embodiments, a subject has or is suspected of having a disease or condition caused by haploinsufficient expression of a target gene. For example a subject that is haploinsufficient for SCN1A expression, in some embodiments, suffers from Dravet syndrome. In some embodiments, an isolated nucleic acid or the rAAV is administered to a subject by intravenous injection, intramuscular injection, inhalation, subcutaneous injection, and/or intracranial injection.

In some aspects, the disclosure provides a composition comprising the isolated nucleic acid or the rAAV as described by the disclosure. In some embodiments, a composition comprises a pharmaceutically acceptable carrier.

In some aspects, the disclosure provides a kit comprising a container housing an isolated nucleic or the rAAV as described by the disclosure. In some embodiments, a kit comprises a container housing a pharmaceutically acceptable carrier. In some embodiments, an isolated nucleic acid or rAAV and a pharmaceutically acceptable carrier are housed in the same container. In some embodiments, a container is a syringe.

In some aspects, the disclosure provides a host cell comprising an isolated nucleic acid or rAAV as described by the disclosure. In some embodiments, a host cell is a eukaryotic cell. In some embodiments, a host cell is a mammalian cell. In some embodiments, a host cell is a human cell, optionally a neuron, for example a GABAergic neuron.

### BRIEF DESCRIPTION OF DRAWINGS

FIG. 1 shows chromatographic sequencing data indicating the sequence conservation between the human (HEK) and mouse (HEPG2) SCN1A genes (Consensus sequence—SEQ ID NO: 98; Target sequence—SEQ ID NO: 99; Hep-SCN1A_R4 sequence (top)—SEQ ID NO: 100; Hep-SCN1A_R4 sequence (bottom)—SEQ ID NO: 101

FIG. 2 shows a sequence alignment of the proximal promoter region of human (SEQ ID NO: 1) and mouse (SEQ ID NO: 2) SCN1A genes, wherein a conserved sequence is highlighted. Within this conserved sequence is a target region of interest for zinc finger protein (ZFP) binding region, which is bolded (SEQ ID NO: 4).

FIG. 3 is a schematic showing the location (SEQ ID NO: 3) of three overlapping target ZFP (ZFP-1, ZFP-2, ZFP-3) (SEQ ID NOs: 5-7) binding sites in the proximal promoter region of the SCN1A gene.

FIGS. 4A-4D shows an alignment of six recognition helix sequences for the individual zinc fingers (Finger 1 through Finger 6; F1-F6) in ZFP-1 which will recognize individual three base regions (DNA triplets denoted in red separated by “⋅”) within the proximal promoter region of the SCN1A gene (SEQ ID NO: 2). FIG. 4A highlights the nucleotide sequence to which zinc fingers 1 through 6 (F1-F6) of ZFP-1 will bind (SEQ ID NO: 3). FIG. 4B shows the three nucleotide sequences recognized by each recognition helix (seven amino acids) of fingers 1 through 6 for ZFP-1 (SEQ ID NOs: 17-22). FIG. 4C shows the amino acid sequences of ZFP-1, which contains 6 fingers, one on each line, wherein the linkers between the fingers are highlighted to designate canonical (TGEKP) and non-canonical (TGSQKP) linker sequences (SEQ ID NOs: 65-70). FIG. 4D shows the nucleotide sequences of ZFP-1 (F1-F6) (SEQ ID NOs: 102-107).

FIGS. 5A-5D shows an alignment of six recognition helix sequences for the individual zinc fingers (Finger 1 through Finger 6; F1-F6) in ZFP-2 which will recognize individual three base regions (DNA triplets denoted in red separated by “*”) within the proximal promoter region of the SCN1A gene (SEQ ID NO: 3). FIG. 5A highlights the nucleotide sequence (SEQ ID NO: 3) to zinc fingers 1 through 6 (F1-F6) of ZFP-2 will bind. FIG. 5B shows the first three nucleotides recognized by each recognition helix (seven amino acids) of fingers 1 through 6 for ZFP-2 (SEQ ID NOs: 29-34). FIG. 5C shows the amino acid sequences of ZFP-2, which contains 6 fingers, one on each line (SEQ ID NOs: 69-74), wherein the linkers between the fingers are highlighted to designate canonical (TGEKP) and non-canonical (TGSQKP) linker sequences. FIG. 5D shows the nucleotide sequences of ZFP-2 (F1-F6) (SEQ ID NOs: 108-113).

FIGS. 6A-6D shows an alignment of six recognition helix sequences for the individual zinc fingers (Finger 1 through Finger 6; F1-F6) in ZFP-3 which will recognize individual three base regions (DNA triplets denoted in red separated by “*”) within the proximal promoter region of the SCN1A gene (SEQ ID NO: 4). FIG. 6A highlights the nucleotide sequence (SEQ ID NO: 3) to zinc fingers 1 through 6 (F1-F6) of ZFP-3 will bind. FIG. 6B shows the first three nucleotides recognized by each recognition helix (seven amino acids) of fingers 1 through 6 for ZFP-3 (SEQ ID NOs: 41-46). FIG. 6C shows the amino acid sequences of ZFP-3, which contains 6 fingers, one on each line (SEQ ID NOs: 75-80), wherein the linkers between the fingers are highlighted to designate canonical (TGEKP) and non-canonical (TGSQKP) linker sequences. FIG. 6D shows the nucleotide sequences of ZFP-3 (F1-F6) (SEQ ID NOs: 114-119).

FIG. 7 shows data indicating that the SCN1A-binding ZFPs described in FIGS. 4-6 increase SCN1A gene expression in HEK293T cells, as measured by quantitative real-time polymerase chain reaction (qRT-PCR). These expression constructs were delivered to the cells via transient transfection of expression plasmids encoding the following transcriptional regulators: Streptococcus pyogenes Cas9+SCN1A guide RNA (SpCas9+Scn1a); Cas9 without endonuclease activity (dCas9); VPR activation domain+SCN1A guide RNA (dCas9_VPR+Scn1a); VPR activation domain+ZFP1 (VPR_ZFP1); VPR activation domain+ZPF2 (VPR_ZFP2); VPR activation domain+ZFP3 (VPR_ZFP3); SpCas9+ASCL1 guide RNA (SpCas9+Ascl1); three VPR_ZFPs (VPR_ZFP1+VPR_ZFP2+VPR_ZFP3). Expression levels were normalized to TBP expression levels determined by qRT-PCR in each sample.

FIG. 8 shows data indicating that the SCN1A-binding ZFPs described in FIGS. 4-6 and Cas9+SCN1A guide RNAs increase SCN1A gene expression in HEK293T cells, as measured by quantitative real-time polymerase chain reaction (qRT-PCR).

### DETAILED DESCRIPTION OF INVENTION

Aspects of the disclosure relate to methods and compositions for modulating (e.g., increasing) expression of a target gene in a cell or subject, wherein the target gene is haploinsufficient (i.e., target gene comprises one functional copy). In some embodiments, the target gene is SCN1A.

In some embodiments, the disclosure provides fusion proteins comprising a DNA binding domain, such as a ZFP, and a transcriptional regulator domain. In some embodiments the disclosure provides fusion proteins comprising a DNA binding domain, such as a ZFP, and a transactivator domain (e.g., a VPR domain). In some embodiments, a DNA binding protein binds to a sequence of target gene or a regulatory region of a target gene. In some embodiments, a regulatory region is an enhancer sequence, a promoter sequence, or a repressor sequence. In some embodiments, a promoter sequence may be an internal promoter (e.g., located in an intron of a target gene) or an external promoter (e.g., located upstream of the transcriptional start site of a target gene). In some embodiments, the DNA binding domain of fusion proteins described herein binds a conserved sequence in the promoter region of a target gene (e.g., SCN1A), whereupon the transactivator domain increases gene expression.

In some aspects, the disclosure relates to methods for increasing expression of a target gene (e.g., SCN1A) in a cell or subject. In some embodiments, the target gene contains mutations which render the cell or subject haploinsufficient for the target gene. Therefore, methods and compositions of the disclosure may be utilized, in some embodiments, to treat diseases and disorders associated with haploinsufficiency of a target gene product, for example Dravet syndrome, which typically results from mutations in one copy of the SCN1A gene leading to haploinsufficiency of the voltage-gated sodium channel alpha subunit Nav1.1.

### Transactivator Fusion Proteins

Some aspects of the disclosure relate to fusion proteins comprising a DNA binding domain (DBD) and a transactivator domain. As used herein, a fusion protein comprises two or more linked polypeptides that are encoded by two or more separate amino acid sequences. Chimeric proteins, as used herein, are fusion proteins wherein the two or more linked genes are from different species. Fusion proteins are typically recombinantly produced, wherein the genes that encode the fusion protein are in a system that supports the expression of the two or more linked genes and the translation of the resulting mRNAs into recombinant proteins. In some embodiments, fusion proteins are recombinantly produced in prokaryotic or eukaryotic cells. Fusion proteins may be configured in multiple arrangements. For example, one protein (Protein A) is located upstream of a second protein (Protein B). In other fusion protein configurations, Protein B is located upstream of Protein A. In some embodiments, a nucleic acid sequence encoding a DNA binding domain is located upstream of a nucleic acid sequence encoding a transactivator domain, and produces a fusion protein comprising the DBD linked to the transactivator. In some embodiments, a nucleic acid sequence encoding a transactivator domain is located upstream of a nucleic acid sequence encoding a DNA binding domain, and produces a fusion protein comprising the transactivator domain linked to the DNA binding domain. In some embodiments, a fusion protein comprises a transactivator domain located upstream of a DNA binding domain. In some embodiments, a fusion protein comprises a DNA binding domain located upstream of a transactivator domain.

In some embodiments, a fusion protein described by the disclosure comprises a DNA binding domain. As used herein, a “DNA binding domain (DBD)” refers to an independently folded protein comprising at least one structural motif which recognizes double- or single-stranded DNA (dsDNA or ssDNA). Certain DBDs recognize specific sequences (recognition sequence or motif), while other types of DBDs have general affinity for DNA. In some embodiments, a fusion protein described by the disclosure comprises a sequence-specific DBD. In some embodiments, the DBD recognizes (e.g., binds specifically to) a nucleic acid sequence within or neighboring the gene encoding a SCN1A protein (e.g., Nav1.1). Proteins containing DBDs are typically involved in cellular processes such as transcription, replication, repair, and DNA storage. The DBDs in transcription factors recognize specific DNA sequences in the promoter region or in enhancer elements to promote gene expression. Transcription factor DBDs are utilized as fusion proteins in genetic engineering to regulate the expression of target genes and can be mutated to alter the DNA binding specificity or DNA binding affinity and thus regulate the expression of a desired target gene. Examples of DBDs include but are not limited to helix-turn-helix motif, zinc finger motifs (including Cys2-His2 zinc fingers), transcription activator-like effectors (TALEs), winged helix motifs, HMG-boxes, dCas proteins (e.g., dCas9 or dCas12a), homeodomains and OB-fold domains.

In some embodiments, the disclosure relates to zinc finger DBD fusion proteins. As used herein, a “zinc finger protein (ZFP)” refers to a protein which contains at least one structural motif characterized by the coordination of one or more zinc ions which stabilize the protein fold. Zinc fingers are among the most diverse structural motifs found in proteins, and up to 3% of human genes encode zinc fingers. Most ZFPs contain multiple zinc fingers which make tandem contacts with target molecules, including DNA, RNA, and the small protein ubiquitin. “Classical” zinc finger motifs are composed of 2 cysteine amino acids and 2 histidine amino acids (C₂H₂) and bind DNA in a sequence-specific manner. These ZFPs, which include transcription factor IIIIA (TFIIIA), are typically involved in gene expression. Multiple zinc finger motifs in DNA binding proteins bind and wrap around the outside of a DNA double helix. Due to their relatively small size (e.g., each finger is about 25-40, usually 27-35 amino acids), zinc finger domain fusion proteins are utilized to create DBDs with novel DNA binding specificity. These DBDs can deliver other fused domains (e.g., transcriptional activation or repression domains or epigenetic modification domains) to alter transcription regulation of a target gene. In some embodiments, zinc finger proteins comprise 2 to 8 fingers, wherein each finger contains 27 to 40 amino acids (e.g., 27, 28, 29, 30, 31, 32, 33, 34, 35, 36, 37, 38, 39 or 40 amino acids).

In some embodiments, a ZFP comprises 1, 2, 3, 4, 5, 6, 7, or 8 zinc fingers. Each zinc finger may comprise 25-40, 25-30, 30-35, 35-40, or 40-45 amino acids. In some embodiments, a zinc finger comprises 27-35 amino acids. In some embodiments, a zinc finger comprises 27, 28, 29, 30, 31, 32, 33, 34, or 35 amino acids. A zinc finger may specifically recognize or bind to a target sequence, e.g., a target gene or a regulatory region of a target gene, that is haploinsufficient in a subject. In some embodiments, a zinc finger binds to a target sequence of a SCN1A gene, e.g., a human SCN1A, for example as set forth in SEQ ID NO: 49. In some embodiments, a zinc finger that binds to a target sequence of a SCN1A gene comprises one or more amino acid sequences of SEQ ID NO: 63-80, or a combination thereof. In some embodiments, a zinc finger specifically recognizes or bind to a target sequence comprising a trinucleotide sequence.

In some embodiments, a zinc finger comprises a recognition helix that recognizes or bind to a target sequence, e.g., a target sequence comprising a trinucleotide sequence. In some embodiments, a recognition helix binds to a trinucleotide In some embodiments, a recognition helix comprises 4-10 amino acids. In some embodiments, a recognition helix comprises 4, 6, 7, 8, 9, or 10 amino acids. In some embodiments, a recognition helix binds to a trinucleotide sequence of a SCN1A gene. In some embodiments, a recognition sequence that binds to a SCN1A gene comprises an amino acid sequence of any one of SEQ ID NO: 17-22, 29-34, or 41-46. In some embodiments, a recognition sequence that binds to a SCN1A gene is encoded by any one of SEQ ID NO: 11-16, 23-28, or 35-40. In some embodiments, a zinc finger binds to the same nucleotide sequence as a recognition helix comprising an amino acid sequence of any one of SEQ ID NO: 17-22, 29-34, or 41-46.

In some embodiments, a zinc finger comprises a linker sequence at its C-terminal end that may serve to link or connect said zinc finger to an additional zinc finger. In some embodiments, a linker sequence may be a canonical linker, e.g., comprising an amino acid sequence of TGEKP (SEQ ID NO: 120). In some embodiments, a linker sequence may be a non-canonical linker, e.g., comprising an amino acid sequence of TGSQKP (SEQ ID NO: 121). In some embodiments, a linker sequence may be 2-10 amino acids, e.g., 2, 3, 4, 5, 6, 7, 8, 9, or 10 amino acids.

In some embodiments, a ZFP that binds to a target gene, e.g., a SCN1A gene, comprises six zinc fingers, each of which recognizes or binds to a different trinucleotide sequence of the target gene, e.g., a SCN1A gene. In some embodiments, a ZFP that binds to a SCN1A gene comprises an amino acid sequence of SEQ ID NO: 57. In some embodiments, a ZFP that binds to a SCN1A gene comprises zinc fingers comprising amino acid sequences of SEQ ID NO: 63, 64, 65, 66, 67, and/or 68. In some embodiments, a ZFP that binds to a SCN1A gene comprises recognition helices comprising amino acid sequences of SEQ ID NO: 17, 18, 19, 20, 21, and/or 22. In some embodiments, a ZFP that binds to a SCN1A gene comprises an amino acid sequence of SEQ ID NO: 59. In some embodiments, a ZFP that binds to a SCN1A gene comprises zinc fingers comprising amino acid sequences of SEQ ID NO: 69, 70, 71, 72, 73, and/or 74. In some embodiments, a ZFP that binds to a SCN1A gene comprises recognition helices comprising amino acid sequences of SEQ ID NO: 29, 30, 31, 32, 33, and/or 34. In some embodiments, a ZFP that binds to a SCN1A gene comprises an amino acid sequence of SEQ ID NO: 61. In some embodiments, a ZFP that binds to a SCN1A gene comprises zinc fingers comprising amino acid sequences of SEQ ID NO: 75, 76, 77, 78, 79, and/or 80. In some embodiments, a ZFP that binds to a SCN1A gene comprises recognition helices comprising amino acid sequences of SEQ ID NO: 41, 42 43, 44, 45, and/or 46. In some embodiments, a ZFP that binds to a SCN1A gene comprises at least 60%, at least 65%, at least 70%, at least 75%, at least 80%, at least 85%, at least 90%, at least 95%, at least 97%, or at least 99% sequence identity to SEQ ID NO: 57, 59, or 61, as shown below.

(amino acid sequence of ZFP1 protein)
SEQ ID NO: 57
RPFQCRICMRNFSQRGNLVRHIRTHTGEKPFACDICGKKFALSFNLTRHT
KIHTGSQKPFQCRICMRNFSRSDNLTRHIRTHTGEKPFACDICGKKFADR
SHLARHTKIHTGSQKPFQCRICMRNFSQKAHLTAHIRTHTGEKPFACDIC
GRKFARSDNLTRHTKIHLRQKD
(amino acid sequence of ZFP2 protein)
SEQ ID NO: 59
RPFQCRICMRNFSRSSNLTRHIRTHTGEKPFACDICGKKFADKRTLIRHT
KIHTGSQKPFQCRICMRNFSQRGNLVRHIRTHTGEKPFACDICGKKFALS
FNLTRHTKIHTGSQKPFQCRICMRNFSRSDNLTRHIRTHTGEKPFACDIC
GRKFADRSHLARHTKIHLRQKD
(amino acid sequence of ZFP3 protein)
SEQ ID NO: 61
RPFQCRICMRNFSDRSALARHIRTHTGEKPFACDICGKKFARSDNLTRHT
KNITGSQKPFQCRICMRNFSQSGDLTRHIRTHTGEKPFACDICGKKFAVR
QTLKQHTKIHTGSQKPFQCRICMRNFSAAGNLTRHIRTHTGEKPFACDIC
GRKFARSDNLTRHTKIHLRQKD

In some embodiments, DBDs are transcription activator-like effector proteins (TALEs). A TALE may specifically recognize or bind to a target sequence, e.g., a target gene or a regulatory region of a target gene. In some embodiments, a subject is haploinsufficient for the target gene. In some embodiments, a TALE binds to a target sequence of a SCN1A gene, e.g., a human SCN1A as provided in SEQ ID NO: 49. TALE proteins are secreted by bacteria and bind promoter sequences in a host plant to activate the expression of plant genes which aid in bacterial infection. Typically, TALE proteins are manipulated to bind new DNA sequences because the recognize target sequences through a central repeat domain consisting of a variable number of ~30-35 amino acid repeats, wherein each repeat recognizes a single base pair within the target sequence. An array of these repeats are typically necessary to recognize a DNA sequence.

In some embodiments, DBDs are homeodomains. A homeodomain may specifically recognize or bind to a target sequence, e.g., a target gene or a regulatory region of a target gene. In some embodiments, a subject is haploinsufficient for the target gene. In some embodiments, a homeodomain binds to a target sequence of a SCN1A gene, e.g., a human SCN1A as provided in SEQ ID NO: 49. Homeodomains are proteins containing three alpha helices and an N-terminal arm that are responsible for recognizing a target sequence. A homeodomain typically recognizes a small DNA sequence (~4 to 8 base pairs), however these domains can be fused in tandem with other DNA-binding domains (either other homeodomains or zinc finger proteins) to recognize longer extended sequences (12 to 24 base pairs). Therefore, homeodomains can be components of DBD that recognize unique sequences within the human genome.

In some embodiments, the at least one DNA binding domain is a catalytically inactive CRISPR associated protein (Cas protein). A catalytically inactive Cas protein (also known as dCas or “dead Cas protein”) is a Cas protein that has been modified or mutated such that it has diminished nuclease activity (e.g., endonuclease activity) or lacks all nuclease activity (e.g., endonuclease activity). In some embodiments, a catalytically inactive Cas protein is a dCas9 or dCas12 protein. In some embodiments, DBDs are dCas proteins (also known as ‘dead Cas’) such as dCas9 or dCas12a. dCas proteins are mutant variants of CRISPR associated proteins (Cas, e.g., Cas9 or Cas12a) that have been mutated such that they are catalytically inactivated, i.e., incapable of nucleotide cleavage. A dCas may specifically recognize or bind to a target sequence, e.g., a target gene or a regulatory region of a target gene. A complex comprising a dCas protein and a guide nucleic acid (e.g., gRNA) can target and/or bind to specific nucleotide sequences or genes that are complementary to the guide nucleic acid. In some embodiments, a subject is haploinsufficient for the target gene. In some embodiments, a dCas binds to a target sequence of a SCN1A gene, e.g., a human SCN1A as provided in SEQ ID NO: 49. However, dCas proteins retain their ability to recognize and bind to target DNA sequences when bound to a guide nucleic acid (e.g., a guide RNA, gRNA, or sgRNA) that is complementary or partially complementary to said target DNA sequence. In some embodiments, a guide nucleic acid for targeting dCas (e.g., dCas9) proteins to SCN1A comprise a spacer sequence having any one of SEQ ID NO: 85, 86, 89, 90, 93, or 94. In some embodiments, a guide nucleic acid for targeting dCas (e.g., dCas9) proteins to SCN1A comprise a spacer sequence having at least 15 (e.g., at least 16, 17, 18, 19, or 20) consecutive nucleotides of any one of SEQ ID NO: 85, 86, 89, 90, 93, or 94. In some embodiments, a guide nucleic acid for targeting dCas (e.g., dCas9) proteins to SCN1A comprises any one of SEQ ID NO: 83, 84, 87, 88, 91, or 92. In some embodiments, a guide nucleic acid for targeting dCas (e.g., dCas9) proteins to SCN1A comprises or consists of any one of SEQ ID NOs: 83-94. Therefore, dCas endonucleases can be components of DBD that recognize unique sequences within the human genome. In some embodiments, a fusion protein comprises a dCas9 protein and a transactivation domain (e.g., a VPR domain).

The disclosure relates, in some aspects, to DNA binding domains that bind to a gene encoding a voltage-gated sodium channel (e.g., Nav1.1). In some embodiments, a gene that encodes a voltage-gated sodium channel is a SCN1A gene, and comprises the sequence set forth in SEQ ID NO: 49. In some embodiments, a DNA binding domain binds to an untranslated region of a target gene, such as a 3′-untranslated region (3′UTR) or a 5′-untranslated region (5′UTR). In some embodiments, an untranslated region comprises a regulatory sequence, for example an enhancer, a promoter, intronic, or a repressor sequence. In some embodiments, a DNA binding domain is a zinc finger protein comprising the sequences set forth in SEQ ID NOs: 57-62. In some embodiments, a DNA binding domain binds to a nucleic acid sequence set forth in any one of SEQ ID NOs: 5-7.

The number of DNA binding domains encoded by a transgene may vary. In some embodiments, a transgene encodes one DNA binding domain. In some embodiments, a transgene encodes 2 DNA binding domains. In some embodiments, a transgene encodes 3 DNA binding domains. In some embodiments, a transgene encodes 4 DNA binding domains. In some embodiments, a transgene encodes 5 DNA binding domains. In some embodiments, a transgene encodes 6 DNA binding domains. In some embodiments, a transgene encodes 7 DNA binding domains. In some embodiments, a transgene encodes 8 DNA binding domains. In some embodiments, a transgene encodes 9 DNA binding domains. In some embodiments, a transgene encodes 10 DNA binding domains. In some embodiments, a transgene encodes more than 10 (e.g., 20, 30, 50, 100, etc.) DNA binding domains. The DNA binding domains may be the same DNA binding domain (e.g., multiple copies of the same DBD), different DNA binding domains (e.g., each DBD binds a unique sequence), or a combination thereof.

The disclosure relates, in some aspects, to fusion proteins comprising a transactivator domain. As used herein, a “transactivation domain” refers to a scaffold domain in a transcription factor which contains binding sites for other proteins which regulate gene expression, such as transcription co-regulators. In some embodiments, a transactivation domain (also known as transcriptional activation domain) acts in conjunction with a DBD to activate transcription from a promoter or enhancer, either directly through contacting transcription factors, or indirectly through coactivator proteins. Transactivation domains (TADs) are commonly named for their amino acid compositions, wherein the amino acids are either essential for activity or are the most abundant in the TAD. TADs are utilized as fusion proteins in genetic engineering to regulate the expression of target genes and can be mutated to alter the level of transcriptional activation and thus expression of the target gene. Examples of transactivation domains include but are not limited to GAL4, HAP1, VP16, P65, RTA, and GCN4.

In some embodiments, a transactivator domain comprises a VP64 domain. VP64 is an acidic TAD composed of four tandem copies of VP16 protein, which is naturally expressed by herpes simplex virus. When fused to a DBD which binds at or near the promoter of a gene, VP64 acts as a strong transcriptional activator and can thus be utilized to regulate expression of a target gene (e.g., SCN1A). The VP64 domain typically consists of a tetrameric repeat of the minimal activation domain of the herpes simplex protein VP16. In some embodiments, the VP64 domain comprises four repeats of amino acid residues 437-448 in VP16. In some embodiments, a VP16 protein is encoded by a human herpes virus 2 UL48 gene, which comprises the sequence set forth in NCBI Ref. Seq. Accession No: NC_001798.2. In some embodiments, a VP16 gene comprises a nucleotide sequence that is 99% identical, 95% identical, 90% identical, 80% identical, 70% identical, 60% identical, or 50% identical to the amino acid sequence encoded by the nucleic acid sequence set forth in NCBI Ref. Seq Accession No: YP_009137200.1. In some embodiments, a VP16 protein comprises an amino acid sequence that is 99% identical, 95% identical, 90% identical, 80% identical, 70% identical, 60% identical, or 50% identical to the amino acid sequence set forth in NCBI Ref. Seq. Accession No Q69113-1. In some embodiments, a VP16 gene comprises a nucleotide sequence that is 99% identical, 95% identical, 90% identical, 80% identical, 70% identical, 60% identical, or 50% identical to the amino acid sequence encoded by the nucleic acid sequence set forth in SEQ ID NO: 51. In some embodiments, a VP16 protein comprises an amino acid sequence that is 99% identical, 95% identical, 90% identical, 80% identical, 70% identical, 60% identical, or 50% identical to the amino acid sequence set forth in SEQ ID NO: 52.

In some embodiments, a transactivator domain comprises a P65 activation domain. P65 is a subunit of the NF-κB transcription factor which contains two adjacent acidic TADs at its C-terminus. When fused to a DBD which binds at or near the promoter of a gene, the p65 protein acts as a strong transcriptional activator and can thus be utilized to regulate expression of a target gene, for example as described by Urlinger, et al. “The p65 domain from NF-kappaB is an efficient human activator in the tetracycline-regulatable gene expression system,” Gene, 2000. In some embodiments, a p65 protein is encoded by a human RELA gene, which comprises the sequence set forth in NCBI Ref. Seq Accession No: NM_001145138.1, NM_001243984.1, NM_001243985.1, or NM_021975.3. In some embodiments, a RELA gene comprises a nucleotide sequence that is 99% identical, 95% identical, 90% identical, 80% identical, 70% identical, 60% identical, or 50% identical to the amino acid sequence encoded by the nucleic acid sequence set forth in any of NCBI Ref. Seq ID Nos: NM_001145138.1, NM_001243984.1, NM_001243985.1, or NM_021975.3. In some embodiments, a p65 protein comprises an amino acid sequence that is 99% identical, 95% identical, 90% identical, 80% identical, 70% identical, 60% identical, or 50% identical to the amino acid sequence set forth in NP_001138610.1, NP_001230913.1, NP_001230914.1, and NP_068110.3. In some embodiments, a RELA gene comprises a nucleotide sequence that is 99% identical, 95% identical, 90% identical, 80% identical, 70% identical, 60% identical, or 50% identical to the amino acid sequence encoded by the nucleic acid sequence set forth in SEQ ID NO: 53. In some embodiments, a p65 protein comprises an amino acid sequence that is 99% identical, 95% identical, 90% identical, 80% identical, 70% identical, 60% identical, or 50% identical to the amino acid sequence set forth in SEQ ID NO: 54.

In some embodiments, a transactivator domain comprises an RTA domain. RTA is a hydrophobic TAD derived from Epstein Barr virus which is a potent transactivation domain which binds to the enhancer region to promote expression of several viral genes. When fused to a DBD which binds at or near the promoter of a gene, the RTA protein acts as a strong transcriptional activator and can thus be utilized to regulate expression of a target gene, for example as described by Miyazawa, et al., “IL-10 promoter transactivation by the viral K-RTA protein involves the host-cell transcription factors, specificity proteins 1 and 3,” Journal of Biological Chemistry, 2018. In some embodiments, a RTA protein is encoded by an Epstein-Barr virus BRLF1 gene, which comprises the sequence set forth in NCBI Ref. Seq Accession No: YP_041674.1. In some embodiments, a BRLF1 gene comprises a nucleotide sequence that is 99% identical, 95% identical, 90% identical, 80% identical, 70% identical, 60% identical, or 50% identical to the amino acid sequence encoded by the nucleic acid sequence set forth in any of NCBI Ref. Seq ID Nos: YP_041674.1. In some embodiments, a RTA protein comprises an amino acid sequence that is 99% identical, 95% identical, 90% identical, 80% identical, 70% identical, 60% identical, or 50% identical to the amino acid sequence set forth in YP_041674.1. In some embodiments, a BRLF1 gene comprises a nucleotide sequence that is 99% identical, 95% identical, 90% identical, 80% identical, 70% identical, 60% identical, or 50% identical to the amino acid sequence encoded by the nucleic acid sequence set forth in SEQ ID NO: 55. In some embodiments, a RTA protein comprises an amino acid sequence that is 99% identical, 95% identical, 90% identical, 80% identical, 70% identical, 60% identical, or 50% identical to the amino acid sequence set forth in SEQ ID NO: 56.

The disclosure is based, in part, on fusion proteins comprising a hybrid transactivator domain. A “hybrid transactivator domain”, as used herein, refers to a fusion protein comprising more than one transcription activating protein or portions thereof (e.g., 2, 3, 4, 5, or more transcription activating proteins, or portions thereof). Hybrid transactivation domains are utilized in genetic engineering to increase the expression of target genes. In some embodiments of the disclosure, a tripartite hybrid transactivation domain comprising the nucleotide sequence for VP64-P65-RTA (VPR), as described in Chavez, et al. “Highly efficient Cas9-mediated transcriptional programming”, Nat Methods, 2015, (SEQ ID NO: 47) is utilized to increase target gene (e.g. SCN1A) expression.

In some embodiments, fusion proteins described herein may comprise a DBD (e.g., a ZFP) and a transcriptional repressor protein. In some aspects, the disclosure relates to fusion proteins comprising a transcriptional repressor domain. A “transcriptional repressor” protein, as used herein, generally refers to a polypeptide which downregulates the expression of a target gene. Examples of transcriptional repressors include, but are not limited to, KRAB, SMRT/TRAC-2, and NCoR/RIP-13. In some embodiments, such transcriptional repressor fusion proteins are useful for reducing the expression level of a target gene (e.g., a gene that is over-expressed in a gain-of-function disease).

### Isolated Nucleic Acids

An isolated nucleic acid sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some aspects, the disclosure relates to isolated nucleic acids (e.g., expression constructs, such as rAAV vectors) that are configured to express one or more ZFP-transactivation domain fusion proteins. In some embodiments, a fusion protein comprises between 1 and 10 (e.g., 1, 2, 3, 4, 5, 6, 7, 8, 9, or 10) DBDs and/or between 1 and 10 (e.g., 1, 2, 3, 4, 5, 6, 7, 8, 9, or 10) transactivator domains. In some embodiments, a fusion protein comprises more than 10 DBS and/or more than 10 transactivator domains.

In some aspects of the disclosure, a DNA binding domains is fused to a transcriptional regulator domain indirectly through a linker. As used herein “a linker” is generally a stretch of polypeptides which structurally join two distinct polypeptides within a single transgene. In some embodiments, a linker is flexible to allow movement of the distinct polypeptides. In some embodiments, a flexible linker comprises glycine residues. In some embodiments, a flexible linker comprises a mixture of glycine and serine residues. In some embodiments, a linker is cleavable, allowing the polypeptides to be separated. In some embodiments, a cleavable linker is cut by a protease. In some embodiments, the protease is trypsin or Factor X.

In some embodiments a linker comprises between 5 and 30 amino acids (e.g., 5, 6, 7, 8, 9, 10, 11, 12, 13, 14, 15, 16, 17, 18, 19, 20, 21, 22, 23, 24, 25, 26, 27, 28, 29, or 30 amino acids). In some embodiments, a linker comprises between 3 and 30 amino acids (e.g., 3, 4, 5, 6, 7, 8, 9, 10, 11, 12, 13, 14, 15, 16, 17, 18, 19, 20, 21, 22, 23, 24, 25, 26, 27, 28, 29, or 30 amino acids). In some embodiments, a linker comprises between 3 and 20 amino acids (e.g., 3, 4 5, 6, 7, 8, 9, 10, 11, 12, 13, 14, 15, 16, 17, 18, 19, or 20 amino acids).

The disclosure is based, in part, on fusion proteins that are engineered to increase expression of a gene encoding a voltage-gated sodium ion channel subunit protein (also referred to as a SCN protein), for example SCN1A. As used herein, “a SCN protein” refers to a sodium ion channel protein which mediates the voltage-dependent sodium ion permeability of excitable membranes, allowing sodium ions to pass through the membrane. Examples of SCN proteins in humans include but are not limited to SCN1A, SCN3A, SCN5A, SCN10A, and SCN11A. In some embodiments, a SCN protein is SCN1A (also referred to as Nav1.1), which encodes a Type 1 α₁ ion channel subunit. In some embodiments, a SCN protein is SCN1B protein, which encodes a Type 1 β₁ ion channel subunit or SCN1C protein. In some embodiments, a SCN protein is a combination of SCN1A, SCN1B, and/or SCN1C proteins. As disclosed herein, a SCN protein can be a portion or a fragment of a SCN protein. In some embodiments, a SCN protein as disclosed herein is a variant of a SCN protein, such as a point mutant or a truncated mutant.

In humans, SCN1A is encoded by the SCN1A gene (Gene ID: 6323, human), which is conserved in chimpanzee, Rhesus monkey, dog, cow, mouse, rat, and chicken. The SCN1A gene in human is primarily expressed in brain, lung, and testis. In some embodiments, SCN1A proteins comprise five structural repeats (I, II, III, IV, Q).

In some embodiments, a SCN1A protein is encoded is encoded by a human SCN1A gene, which comprises the sequence set forth in NCBI Ref. Seq ID No: NM_001165963.2, NM_00165964.2, NM_001202435.2, NM_001353948.1, NM_001353949.1, NM_001353950.1, NM_00135395.1, NM_001353952.1, NM_001353954.1, NM_00353955.1, NM_001353957.1, NM_001353958.1, NM_001353960.1, NM_001353961.1, or NM_006920.5. In some embodiments, a SCN1A protein is encoded by a mouse SCN1A gene, which comprises the sequence set forth in NCBI Ref Seq ID No: NM_001313997.1 or NM_018733.2. In some embodiments, a SCN1A protein comprises an amino acid sequence that is 99% identical, 95% identical, 90% identical, 80% identical, 70% identical, 60% identical, or 50% identical to the amino acid sequence encoded by the nucleic acid sequence set forth in either NCBI Ref. Seq ID No: NG_011906.1, NM_001313997.1 or NM_018733.2. In some embodiments, a SCN1A gene comprises an amino acid sequence that is 99% identical, 95% identical, 90% identical, 80% identical, 70% identical, 60% identical, or 50% identical to the sequence set forth in SEQ ID NO: 50. In some embodiments, a human SCN1A protein comprises the sequence set forth in NCBI Ref. Seq ID No: NP_001159435.1, NP_0011159436.1, NP_001189364.1, NP_001340877.1, NP_001340878.1, NP_001340879.1, NP_001340880.1, NP_001340881.1, NP_001340883.1, NP_001340884.1, NP_001340886.1, NP_001340887.1, NP_001340889.1, NP_001340890.1, NP_00851.3. In some embodiments, a SCN1A protein comprises an amino acid sequence that is 99% identical, 95% identical, 90% identical, 80% identical, 70% identical, 60% identical, or 50% identical to the amino acid sequence encoded by the nucleic acid sequence set forth in either NCBI Ref. Seq ID No: NG_011906.1, NM_001313997.1 or NM_018733.2. In some embodiments, a mouse SCN1A protein comprises the sequence set forth in NCBI Ref. Seq ID No: NP_001300926.1 or NP_061203.2. In some embodiments, a human SCN1A protein comprises an amino acid sequence that is 99% identical, 95% identical, 90% identical, 80% identical, 70% identical, 60% identical, or 50% identical to the nucleic acid sequence set forth in SEQ ID NO: 49.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., comprising a fusion protein, etc.) may be positioned at any suitable location of the isolated nucleic acid that will enable expression of the fusion protein.

It should be appreciated that in cases where a transgene encodes more than one polypeptide, each polypeptide may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is a P2 promoter. In some embodiments, a promoter is a chicken β-actin (CBA) promoter. In some embodiments, a promoter is two CBA promoters. In some embodiments, a promoter is two CBA promoters separated by a CMV enhancer. In some embodiments, a promoter is a CAG promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, a transgene which encodes a fusion protein comprising a DBD and a transactivator is operably linked to a promoter. In some embodiments, the promoter is a constitutive promoter. In some embodiments, the promoter is an inducible promoter. In some embodiments, the promoter is a tissue-specific promoter. In some embodiments, the promoter is specific for nervous tissue. In some embodiments, the promoter is SST or NPY promoter.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding a protein it may be desirable to drive expression of the first protein coding region using a first promoter sequence (e.g., a first promoter sequence operably linked to the protein coding region), and to drive expression of the second protein coding region with a second promoter sequence (e.g., a second promoter sequence operably linked to the second protein coding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (pol III) promoter sequence. Non-limiting examples of pol III promoter sequences include U6 and H1 promoter sequences. In some embodiments, the second promoter sequence (e.g., the promoter sequence driving expression of the second protein) is a RNA polymerase II (pol II) promoter sequence. Non-limiting examples of pol II promoter sequences include T7, T3, SP6, RSV, and cytomegalovirus promoter sequences. In some embodiments, a pol III promoter sequence drives expression of the first protein coding region. In some embodiments, a pol II promoter sequence drives expression of the second protein coding region.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, and AAV.PHP.B. In some embodiments, an AAV capsid protein is of a serotype derived from a non-human primate, for example AAVrh8 serotype. In some embodiments, an AAV capsid protein is of a serotype derived for broad and efficient CNS transduction, for example AAV.PHP.B. In some embodiments, the capsid protein is of AAV serotype 9.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a transgene (e.g., a DNA binding domain fused to a transcriptional regulator domain). In some embodiments, the host cell is a mammalian cell, a yeast cell, a bacterial cell, an insect cell, a plant cell, or a fungal cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a neuron, optionally a GABAergic neuron. A “GABAergic neuron”, as used herein, is a neural cell that generates gamma aminobutyric acid (GABA). In mammals, GABA is a neurotransmitter that is widely distributed in the nervous system which binds and represses the neurons which it binds. As such, GABA is implicated in numerous disorders affecting the nervous system, including epilepsy, autism, and anxiety. Studies in SCN1A hemizygote and knock-out mice have observed a profound sodium current deficit in GABAergic neurons in the brain. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

A “promoter” refers to a DNA sequence recognized by the synthetic machinery of the cell, or introduced synthetic machinery, required to initiate the specific transcription of a gene. The phrases “operatively linked”, “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product from a transcribed gene. The foregoing methods for packaging recombinant vectors in desired AAV capsids to produce the rAAVs of the disclosure are not meant to be limiting and other suitable methods will be apparent to the skilled artisan.

### Methods for Regulating Target Gene Expression

Methods for regulating gene expression in a cell or subject are provided by the disclosure. The methods typically involve administering to a cell or a subject an isolated nucleic acid or rAAV comprising a transgene which encodes a fusion protein comprising a DNA binding domain (e.g., a ZFP domain) and a transactivation domain. In some embodiments, a fusion protein comprises ZFP and VP64 transactivator. In some embodiments, a fusion protein comprises ZFP and p65 transactivator. In some embodiments, a fusion protein comprises ZFP and RTA transactivator. In some embodiments, a fusion protein comprises ZFP and VPR transactivator. In some embodiments, the method involves administering to a cell or a subject a dCas9 protein and at least one guide nucleic acid that targets SCN1A (e.g., a guide nucleic acid comprising any one of SEQ ID NO: 83-94 or encoded by any one of SEQ ID NO: 83-94).

Administering an isolated nucleic acid or an rAAV encoding the fusion protein (e.g., a fusion protein comprising a transactivator) to a cell or subject, in some embodiments, results in increased expression of a target gene (e.g., SCN1A). Thus, in some embodiments, compositions and methods described by the disclosure are useful for treating conditions resulting from a haploinsufficiency of a target gene, such as Dravet syndrome which results from haploinsufficiency of SCN1A gene.

As used herein, a “haploinsufficiency” refers to a genetic condition wherein one copy of a gene (e.g., SCN1A) is inactivated, e.g., by genetic mutation, or deleted, and the remaining functional copy of the gene is not adequate to produce an amount of gene product sufficient to preserve normal function of the gene.

Dravet syndrome, also known as Severe Myoclonic Epilepsy of Infancy, is a rare, life-long form of epilepsy which typically manifests in the first three years of life. Dravet syndrome is characterized by prolonged and frequent seizures, behavioral and developmental delays, movement and balance issues, delayed language and speech issues, and disruptions of the autonomic nervous system. In some embodiments, a subject has a haploinsufficiency associated with Dravet syndrome, such as one copy of the SCN1A gene being mutated, resulting in reduced SCN1A protein in a cell or subject. The majority of Dravet syndrome patients carry SCN1A mutations which are translated into truncated proteins; other SCN1A mutations associated with Dravet syndrome include splice-site and missense mutations, as well as mutations randomly distributed throughout the SCN1A gene. In some embodiments, a fusion protein of the disclosure comprises a ZFP domain that specifically targets (e.g., binds to) a SCN1A gene and a transactivation domain. In some embodiments, a composition for targeting SCNA1 comprises (i) a fusion protein comprising a dCas protein and a transactivation domain, and (ii) a guide nucleic acid (e.g., a gRNA) that specifically targets (e.g., binds to) a SCN1A gene.

In some embodiments, a subject has a haploinsufficiency associated with MED13L haploinsufficiency syndrome, wherein the subject only has a single functional copy of the MED13L gene. Subjects suffering from MED13L haploinsufficiency syndrome typically have a mutation in their second, non-functional copy of the MED13L gene. MED13L haploinsufficiency syndrome is characterized by intellectual disability, speech problems, distinctive facial features, and developmental delay. In some embodiments, a fusion protein of the disclosure comprises a ZFP domain that specifically targets (e.g., binds to) a MED13L gene and a transactivation domain. In some embodiments, a composition for targeting MED13L comprises (i) a fusion protein comprising a dCas protein and a transactivation domain, and (ii) a guide nucleic acid (e.g., a gRNA) that specifically targets (e.g., binds to) a MED13L gene.

In some embodiments, a subject has a haploinsufficiency associated with myelodysplastic syndromes. Subjects suffering from a myelodysplastic syndrome typically have a mutation in one copy of the isocitrate dehydrogenase 1 (IDH1), isocitrate dehydrogenase 2 (IDH2), and/or GATA2 genes. Myelodysplastic syndrome are a group of cancers in which immature blood cells in the bone marrow do not mature into healthy blood cells. Occasionally, this syndrome can lead to acute myeloid leukemia. In some embodiments, a fusion protein of the disclosure comprises a ZFP domain that specifically targets (e.g., binds to) an IDH1 gene and a transactivation domain. In some embodiments, a composition for targeting IDH1 comprises (i) a fusion protein comprising a dCas protein and a transactivation domain, and (ii) a guide nucleic acid (e.g., a gRNA) that specifically targets (e.g., binds to) a IDH1 gene. In some embodiments, a fusion protein of the disclosure comprises a ZFP domain that specifically targets (e.g., binds to) an IDH2 gene and a transactivation domain. In some embodiments, a composition for targeting IDH2 comprises (i) a fusion protein comprising a dCas protein and a transactivation domain, and (ii) a guide nucleic acid (e.g., a gRNA) that specifically targets (e.g., binds to) a IDH2 gene. In some embodiments, a fusion protein of the disclosure comprises a ZFP domain that specifically targets (e.g., binds to) an GATA2 gene and a transactivation domain. In some embodiments, a composition for targeting GATA2 comprises (i) a fusion protein comprising a dCas protein and a transactivation domain, and (ii) a guide nucleic acid (e.g., a gRNA) that specifically targets (e.g., binds to) a GATA2 gene.

In some embodiments, a subject has a haploinsufficiency associated with DiGeorge syndrome. Subjects suffering from a DiGeorge syndrome typically have a deletion of 30 to 40 genes in the middle of chromosome 22 at a location known as 22q11.2. In particular, the disease may be characterized by haploinsufficiency of the TBX gene. DiGeorge syndrome is characterized by congenital heart problems, specific facial features, frequent infections, developmental delay, learning problems and cleft palate. In some embodiments, a fusion protein of the disclosure comprises a ZFP domain that specifically targets (e.g., binds to) a TBX gene and a transactivation domain. In some embodiments, a composition for targeting TBX comprises (i) a fusion protein comprising a dCas protein and a transactivation domain, and (ii) a guide nucleic acid (e.g., a gRNA) that specifically targets (e.g., binds to) a TBX gene.

In some embodiments, a subject has a haploinsufficency associated with CHARGE syndrome. In a majority of cases, subjects suffering from CHARGE syndrome are haploinsufficient for the CHD7 gene. CHARGE syndrome is characterized by coloboma of the eye, heart defects, atresia of the nasal choanae, retardation of growth and/or development, genital and/or urinary abnormalities, and ear abnormalities and deafness. In some embodiments, a fusion protein of the disclosure comprises a ZFP domain that specifically targets (e.g., binds to) a CHD7 gene and a transactivation domain. In some embodiments, a composition for targeting CHD7 comprises (i) a fusion protein comprising a dCas protein and a transactivation domain, and (ii) a guide nucleic acid (e.g., a gRNA) that specifically targets (e.g., binds to) a CHD7 gene.

In some embodiments, a subject has a haploinsufficency associated with Ehlers-Danlos syndrome. Subjects suffering from Ehlers-Danlos syndrome may be haploinsufficient for the COL1A1, COL1A2, COL3A1, COL5A1, COL5A2, TNXB, ADAMTS2, PLOD1, B4GALT7, DSE, and/or D4ST1/CHST14 genes. Ehlers-Danlos syndrome is characterized by skin hyperelasticity and may result in aortic dissection, scoliosis, and early-onset osteoarthritis. In some embodiments, a fusion protein of the disclosure comprises a ZFP domain that specifically targets (e.g., binds to) any one of COL1A1, COL1A2, COL3A1, COL5A1, COL5A2, TNXB, ADAMTS2, PLOD1, B4GALT7, DSE, or D4ST1/CHST14 genes and a transactivation domain. In some embodiments, a composition for targeting any one of COL1A1, COL1A2, COL3A1, COL5A1, COL5A2, TNXB, ADAMTS2, PLOD1, B4GALT7, DSE, or D4ST1/CHST14 comprises (i) a fusion protein comprising a dCas protein and a transactivation domain, and (ii) a guide nucleic acid (e.g., a gRNA) that specifically targets (e.g., binds to) any one of COL1A1, COL1A2, COL3A1, COL5A1, COL5A2, TNXB, ADAMTS2, PLOD1, B4GALT7, DSE, or D4ST1/CHST14 gene.

In some embodiments, a subject has a haploinsufficency associated with frontotemporal dementias (FTD). Subjects suffering from FTD are haploinsufficient for the MAPT gene, which encodes Tau protein, and/or the GRN gene. FTD is characterized by memory loss, lack of social awareness, poor impulse control, and difficulties in speech. In some embodiments, a fusion protein of the disclosure comprises a ZFP domain that specifically targets (e.g., binds to) a MAPT gene and a transactivation domain. In some embodiments, a composition for targeting MAPT comprises (i) a fusion protein comprising a dCas protein and a transactivation domain, and (ii) a guide nucleic acid (e.g., a gRNA) that specifically targets (e.g., binds to) a MAPT gene. In some embodiments, a fusion protein of the disclosure comprises a ZFP domain that specifically targets (e.g., binds to) a GRN gene and a transactivation domain. In some embodiments, a composition for targeting GRN comprises (i) a fusion protein comprising a dCas protein and a transactivation domain, and (ii) a guide nucleic acid (e.g., a gRNA) that specifically targets (e.g., binds to) a GRN gene.

In some embodiments, a subject has a haploinsufficency associated with Holt-Oram syndrome. Subjects suffering from Holt-Oram syndrome are haploinsufficient for the TBX5 gene. Holt-Oram syndrome is characterized by heart complications, including congenital heart defects and cardiac conduction disease. In some embodiments, a fusion protein of the disclosure comprises a ZFP domain that specifically targets (e.g., binds to) a TBX5 gene and a transactivation domain. In some embodiments, a composition for targeting TBX5 comprises (i) a fusion protein comprising a dCas protein and a transactivation domain, and (ii) a guide nucleic acid (e.g., a gRNA) that specifically targets (e.g., binds to) a TBX5 gene.

In some embodiments, a subject has a haploinsufficency associated with Marfan syndrome. Subjects suffering from Marfan syndrome are typically haploinsufficient for the FBN1 gene, encoding fibrillin-1 protein. Marfan syndrome is characterized by disproportionate limb lengths, early-onset arthritis, heart complications, and/or dysfunction of the autonomic nervous system. In some embodiments, a fusion protein of the disclosure comprises a ZFP domain that specifically targets (e.g., binds to) a FBN1 gene and a transactivation domain. In some embodiments, a composition for targeting FBN1 comprises (i) a fusion protein comprising a dCas protein and a transactivation domain, and (ii) a guide nucleic acid (e.g., a gRNA) that specifically targets (e.g., binds to) a FBN1 gene.

The disclosure is based, in part, on methods of administering a fusion protein as described herein to a subject. In some embodiments, the fusion protein comprises a DBD and a transcriptional activator. In some embodiments, the DBD is a ZNF, a TALE, a dCas protein (e.g., dCas9 or dCas12a), or a homeodomain that binds to a SCN1A gene. In some embodiments, the transcriptional activator is VP64, p65, RTA, or a tripartite transcription activator comprising VP64-p65-RTA (VPR). In some embodiments, the fusion protein is flanked by AAV inverted terminal repeat (ITR) sequences. In some embodiments, the fusion protein is operably linked to a promoter. In some embodiments, the subject has or is suspected of having mutations in SCN1A that result in SCN1A protein haploinsufficiency. In some embodiments, the subject has or is suspected of having Dravet syndrome.

In some aspects, the disclosure provides methods of modulating (e.g., increasing, decreasing, etc.) expression of a target gene in a cell. In some embodiments, the disclosure provides methods of increasing expression of a target gene (e.g., SCN1A) in a cell. In some embodiments, a cell is a mammalian cell. In some embodiments, a cell is in a subject (e.g., in vivo). In some embodiments, a subject is a mammalian subject, for example a human. In some embodiments, a cell is a nervous system cell (central nervous system cell or peripheral nervous system cell), for example a neurons (e.g., GABAergic neurons, unipolar neurons, bipolar neurons, Basket cells, Betz cells, Lugaro cells, spiny neurons, Purkinje cells, Pyrimidal cells, Renshaw cells, Granule cells, motor neurons, spindle cells, etc.) or glial cells (e.g., astrocytes, oligodendrocytes, ependymal cells, radial glia, Schwann cells, Satellite cells, etc.).

In a “normal” cell or subject, the expression of a target gene (e.g., SCN1A) is sufficient such that cell or subject is not haploinsufficient with regard to the target gene (e.g., SCN1A). In some embodiments, “improved” or “increased” expression or activity of a transgene is measured relative to expression or activity of that transgene in a cell or subject who has not been administered one or more isolated nucleic acids, rAAVs, or compositions as described herein. In some embodiments, “improved” or “increased” expression or activity of a transgene is measured relative to expression or activity of that transgene in the subject after the subject has been administered (e.g., gene expression is measured pre- and post-administration of) one or more isolated nucleic acids, rAAVs, or compositions as described herein For example, in some embodiments, “improved” or “increased” expression of SCN1A in a cell or subject is measured relative to a cell or subject who has not been administered a transgene encoding a fusion ZFP-transactivator. In some embodiments, methods described by the disclosure result in SCN1A expression and/or activity in a subject that is increased between 2-fold and 100-fold (e.g., 2-fold, 5-fold, 10-fold, 50-fold, 100-fold, etc.) relative to the SCN1A expression and/or activity of a subject who has not been administered one or more compositions described by the disclosure.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms, for example, symptoms associated with a haploinsufficient gene, e.g., a haploinsufficent SCN1A gene. Thus, the terms denote that a beneficial result has been conferred on a subject with a disorder (e.g., a disease or condition associated with a haploinsufficient gene, e.g., Dravet syndrome), or with the potential to develop such a disorder. Furthermore, the term “treatment” also includes the application or administration of an agent (e.g., therapeutic agent or a therapeutic composition, e.g., an isolated nucleic acid or rAAV that targets or binds to a target gene or a regulatory region of a target gene) to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease.

Therapeutic agents or therapeutic compositions may include a compound in a pharmaceutically acceptable form that prevents and/or reduces the symptoms of a particular disease (e.g., a disease or condition associated with a haploinsufficient gene, e.g., Dravet syndrome). For example a therapeutic composition may be a pharmaceutical composition that prevents and/or reduces the symptoms of a disease or condition associated with a haploinsufficient gene, e.g., Dravet syndrome. It is contemplated that the therapeutic composition of the present invention will be provided in any suitable form. The form of the therapeutic composition will depend on a number of factors, including the mode of administration as described herein. The therapeutic composition may contain diluents, adjuvants and excipients, among other ingredients as described herein.

### Modes of Administration

The isolated nucleic acids, rAAVs and compositions of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), thalamus, spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, an rAAV as described in the disclosure are administered by intravenous injection. In some embodiments, rAAVs are administered by intracerebral injection. In some embodiments, rAAVs are administered by intrathecal injection. In some embodiments, rAAVs are administered by intrastriatal injection. In some embodiments, rAAVs are delivered by intracranial injection. In some embodiments, rAAVs are delivered by cisterna magna injection. In some embodiments, the rAAV are delivered by cerebral lateral ventricle injection.

Aspects of the instant disclosure relate to compositions comprising a recombinant AAV comprising a capsid protein and a nucleic acid encoding a transgene, wherein the transgene comprises a nucleic acid sequence encoding one or more proteins. In some embodiments, the nucleic acid further comprises AAV ITRs. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is administered to the subject during a pre-symptomatic stage of the lysosomal storage disease. In some embodiments, the pre-symptomatic stage of the lysosomal storage disease occurs between birth (e.g., perinatal) and 4-weeks of age.

In some embodiments, rAAV compositions are formulated to reduce aggregation of AAV particles in the composition, particularly where high rAAV concentrations are present (e.g., ~10¹³ GC/mL or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

### Example 1. Design of Zinc Finger Proteins to Upregulate SCN1A Gene Expression

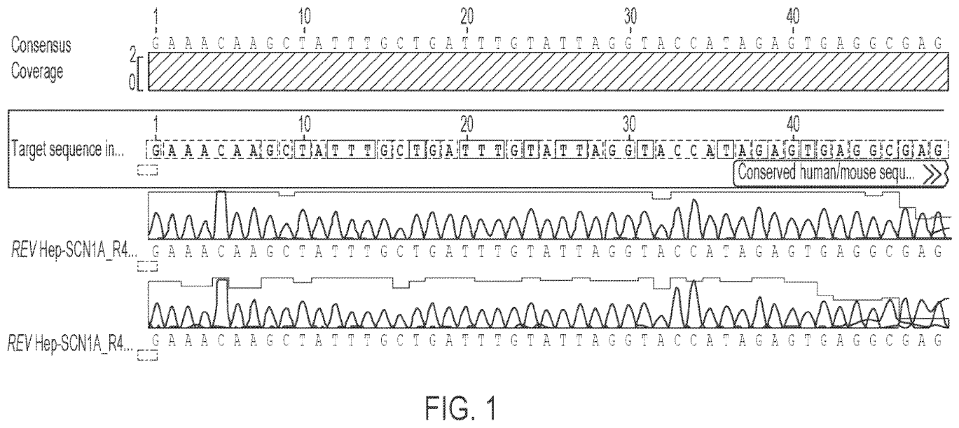

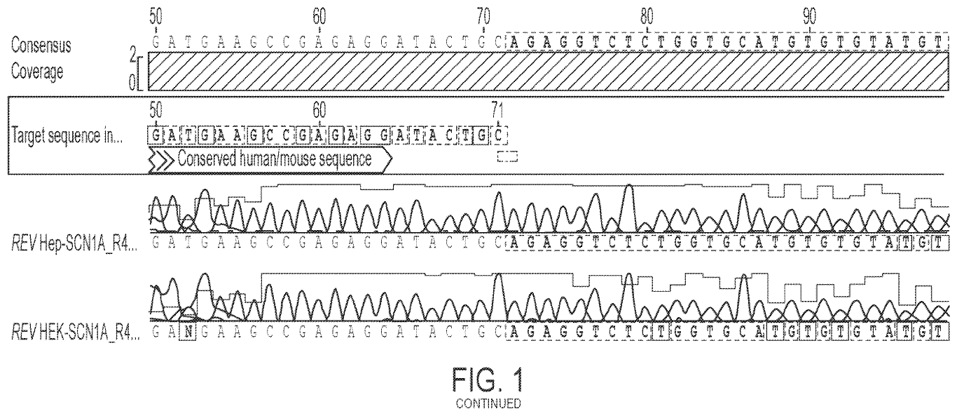

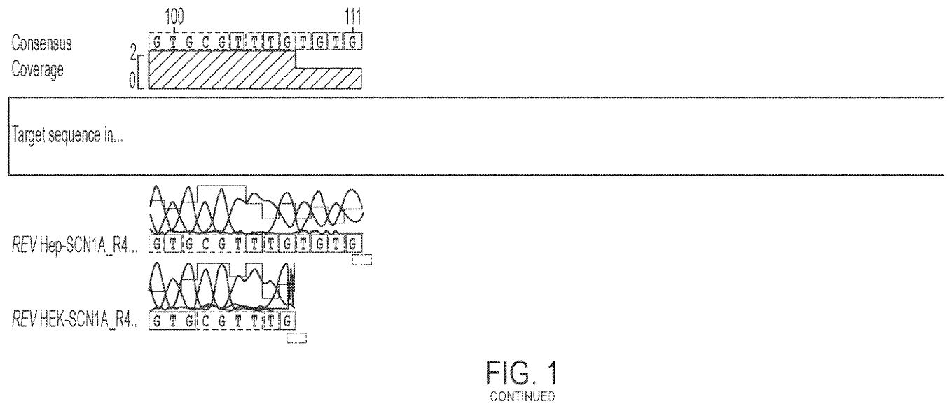

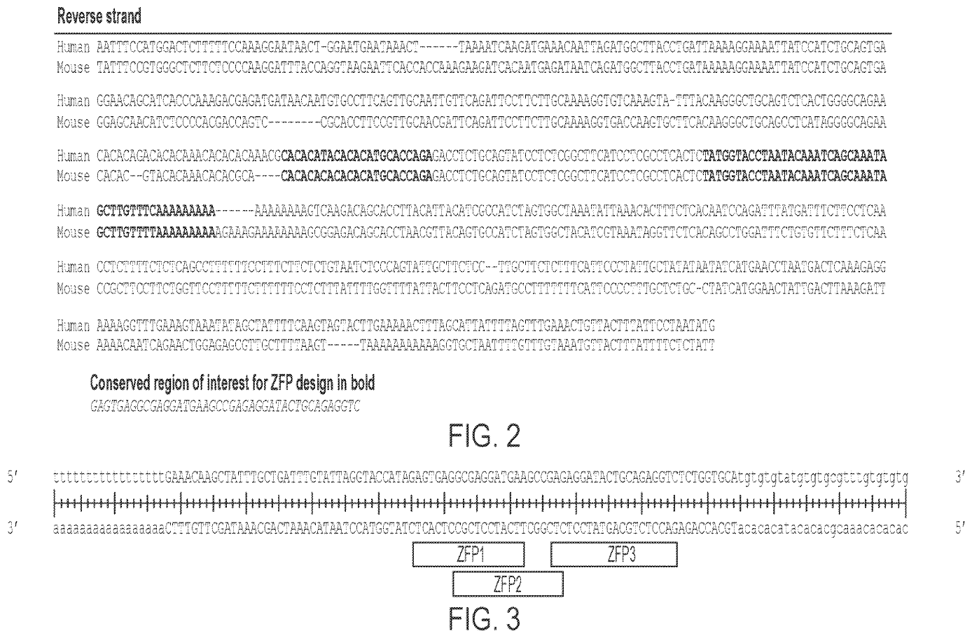

Homologous regions between the human (HEK293T cells) and mouse (HEPG2 cells) SCN1A promoter sequences were identified by alignment of sequences surrounding the two prominent transcription start sites identified in the RIKEN CAGE-seq data set for each species (FIG. 1). A highly conserved sequence between human (HEK) and mouse (HEPG2) exists in the proximal promoter region of SCN1A (FIG. 2). Three ZFPs consisting of six fingers were designed to bind overlapping 15-22 nucleotide regions of homology in the proximal promoter region of SCN1A through the assembly of one and two-finger modules with pre-defined DNA-binding specificity (FIG. 3). Three ZFPs (ZFP1-ZFP3) consisting of six fingers each were designed to bind the overlapping highly conserved sequences identified in FIG. 3. Each finger is designed to bind a three base region (triplet) in the highly conserved region of the proximal promoter of SCN1A.

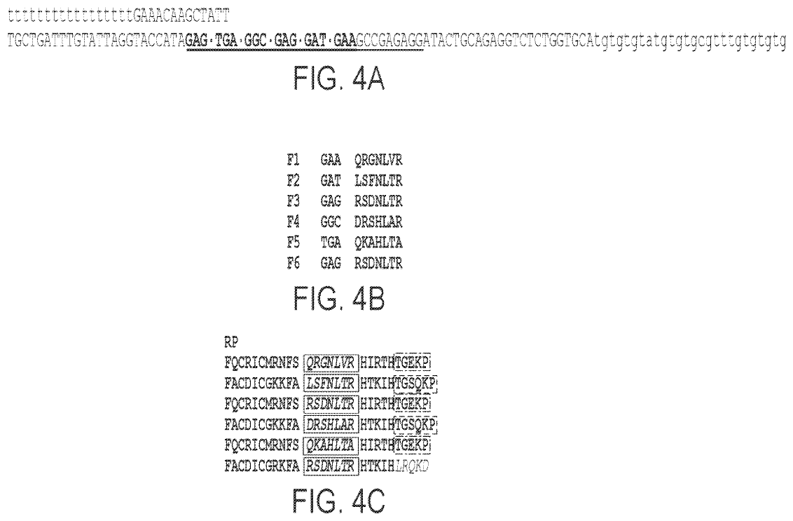

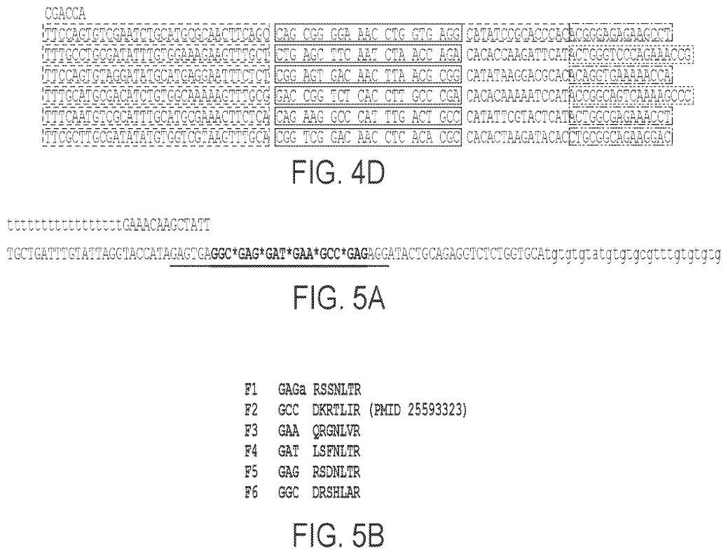

ZFP-1 recognizes individual three base regions (DNA triplets denoted in red separated by “⋅”) within the proximal promoter region of the SCN1A gene (SEQ ID NO: 2), as shown in FIG. 4A. Each recognition helix (seven amino acids) of fingers 1 through 6 for ZFP-1 bind a three nucleotide sequences, as shown in FIG. 4B. The amino acid sequences of the six fingers of ZFP-1 (SEQ ID NOs: 17-22) are shown in FIG. 4C; the linkers between the fingers are highlighted to designate canonical (TGEKP) and non-canonical (TGSQKP) linker sequences. Nucleotide sequences of the six fingers of ZFP-1 (SEQ ID NOs: 11-16) are shown in FIG. 4D.

TABLE 1
Recognition helices of ZFP-1 that targets SCN1A

| | Amino Acid Sequence | Nucleotide Sequence |
|---|---|---|
| ZFP-1 Recognition Helix 1 | QRGNLVR | CAGCGGGGAAACCTGGTGAGG |
| | (SEQ ID NO: 17) | (SEQ ID NO: 11) |
| ZFP-1 Recognition Helix 2 | LSFNLTR | CTGAGCTTCAATCTAACCAGA |
| | (SEQ ID NO: 18) | (SEQ ID NO: 12) |
| ZFP-1 Recognition Helix 3 | RSDNLTR | CGGAGTGACAACTTAACGCGG |
| | (SEQ ID NO: 19) | (SEQ ID NO: 13) |
| ZFP-1 Recognition Helix 4 | DRSHLAR | GACCGGTCTCACCTTGCCCGA |
| | (SEQ ID NO: 20) | (SEQ ID NO: 14) |
| ZFP-1 Recognition Helix 5 | QKAHLTA | CAGAAGGCCCATTTGACTGCC |
| | (SEQ ID NO: 21) | (SEQ ID NO: 15) |
| ZFP-1 Recognition Helix 6 | RSDNLTR | CGGTCGGACAACCTCACACGC |
| | (SEQ ID NO: 22) | (SEQ ID NO: 16) |

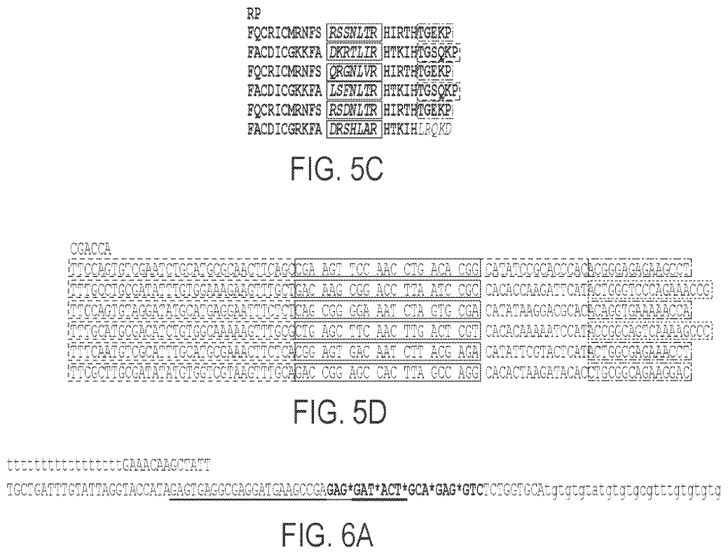

ZFP-2 recognizes individual three base regions (DNA triplets denoted in red separated by “⋅”) within the proximal promoter region of the SCN1A gene (SEQ ID NO: 3), as shown in FIG. 5A. Each recognition helix (seven amino acids) of fingers 1 through 6 for ZFP-2 bind a three nucleotide sequences, as shown in FIG. 5B. The amino acid sequences of the six fingers of ZFP-2 (SEQ ID NOs: 29-34) are shown in FIG. 5C; the linkers between the fingers are highlighted to designate canonical (TGEKP) and non-canonical (TGSQKP) linker sequences. Nucleotide sequences of the six fingers of ZFP-1 (SEQ ID NOs: 23-28) are shown in FIG. 5D.

TABLE 2
Recognition helices of ZFP-2 that targets SCN1A

| | Amino Acid Sequence | Nucleotide Sequence |
|---|---|---|
| ZFP-2 Recognition Helix 1 | RSSNLTR | CGAAGTTCCAACCTGACACGG |
| | (SEQ ID NO: 29) | (SEQ ID NO: 23) |
| ZFP-2 Recognition Helix 2 | DKRTLIR | GACAAGCGGACCTTAATCCGC |
| | (SEQ ID NO: 30) | (SEQ ID NO: 24) |
| ZFP-2 Recognition Helix 3 | QRGNLVR | CAGCGGGGAAATCTAGTGCGA |
| | (SEQ ID NO: 31) | (SEQ ID NO: 25) |
| ZFP-2 Recognition Helix 4 | LSFNLTR | CTGAGCTTCAACTTGACTCGT |
| | (SEQ ID NO: 32) | (SEQ ID NO: 26) |
| ZFP-2 Recognition Helix 5 | RSDNLTR | CGGAGTGACAATCTTACGAGA |
| | (SEQ ID NO: 33) | (SEQ ID NO: 27) |
| ZFP-2 Recognition Helix 6 | DRSHLAR | GACCGGAGCCACTTAGCCAGG |
| | (SEQ ID NO: 34) | (SEQ ID NO: 28) |

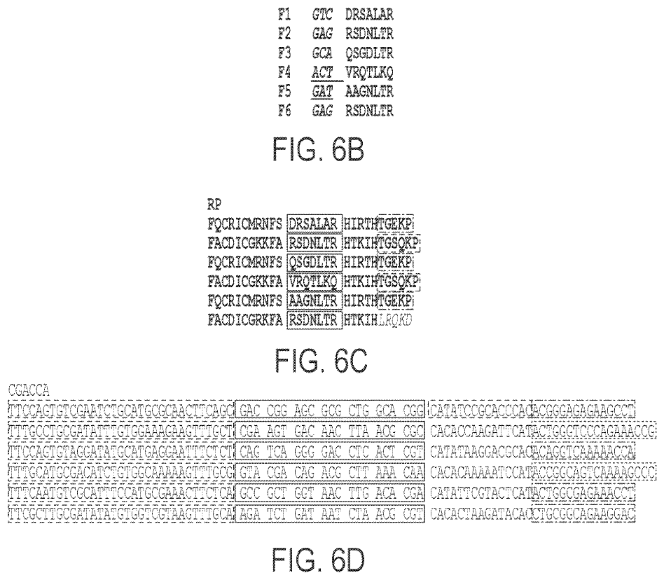

ZFP-3 recognizes individual three base regions (DNA triplets denoted in red separated by “⋅”) within the proximal promoter region of the SCN1A gene (SEQ ID NO: 4), as shown in FIG. 6A. Each recognition helix (seven amino acids) of fingers 1 through 6 for ZFP-3 bind a three nucleotide sequences, as shown in FIG. 6B. The amino acid sequences of the six fingers of ZFP-3 (SEQ ID NOs: 41-46) are shown in FIG. 6C; the linkers between the fingers are highlighted to designate canonical (TGEKP) and non-canonical (TGSQKP) linker sequences. Nucleotide sequences of the six fingers of ZFP-1 (SEQ ID NOs: 35-40) are shown in FIG. 6D.

TABLE 3
Recognition helices of ZFP-3 that targets SCN1A

| | Amino Acid Sequence | Nucleotide Sequence |
|---|---|---|
| ZFP-3 Recognition Helix 1 | DRSALAR | GACCGGAGCGCGCTGGCACGG |
| | (SEQ ID NO: 41) | (SEQ ID NO: 35) |
| ZFP-3 Recognition Helix 2 | RSDNLTR | CGAAGTGACAACTTAACGCGC |
| | (SEQ ID NO: 42) | (SEQ ID NO: 36) |
| ZFP-3 Recognition Helix 3 | QSGDLTR | CAGTCAGGGGACCTCACTCGT |
| | (SEQ ID NO: 43) | (SEQ ID NO: 37) |
| ZFP-3 Recognition Helix 4 | VRQTLKQ | GTACGACAGACGCTTAAACAA |
| | (SEQ ID NO: 44) | (SEQ ID NO: 38) |
| ZFP-3 Recognition Helix 5 | AAGNLTR | GCCGCTGGTAACTTGACACGA |
| | (SEQ ID NO: 45) | (SEQ ID NO: 39) |
| ZFP-3 Recognition Helix 6 | RSDNLTR | AGATCTGATAATCTAACGCGT |
| | (SEQ ID NO: 46) | (SEQ ID NO: 40) |

Additional ZFPs designed to target sequences conserved in the proximal promoter region of the SCN1A gene will comprise five or six finger domains each and will bind to regions of 15-22 nucleotides that are highly conserved between human and mouse SCN1A.

TABLE 4
Zinc finger proteins that target SCN1A

| | Amino Acid Sequence | Nucleotide Sequence |
|---|---|---|
| ZFP-1 | RPFQCRICMRNFSQRGNLV | CGACCATTCCAGTGTCGAATCTGCATGCGCAA |
| | RHIRTHTGEKPFACDICGKK | CTTCAGCCAGCGGGGAAACCTGGTGAGGCAT |
| | FALSFNLTRHTKIHTGSQKP | ATCCGCACCCACACGGGAGAGAAGCCTTTTGC |
| | FQCRICMRNFSRSDNLTRHI | CTGCGATATTTGTGGAAAGAAGTTTGCTCTGA |
| | RTHTGEKPFACDICGKKFA | GCTTCAATCTAACCAGACACACCAAGATTCAT |
| | DRSHLARHTKIHTGSQKPF | ACTGGGTCCCAGAAACCGTTCCAGTGTAGGAT |
| | QCRICMRNFSQKAHLTAHI | ATGCATGAGGAATTTCTCTCGGAGTGACAACT |
| | RTHTGEKPFACDICGRKFA | TAACGCGGCATATAAGGACGCACACAGGTGA |
| | RSDNLTRHTKIHLRQKD | AAAACCATTTGCATGCGACATCTGTGGCAAAA |
| | (SEQ ID NO: 57) | AGTTTGCGGACCGGTCTCACCTTGCCCGACAC |
| | | ACAAAAATCCATACCGGCAGTCAAAAGCCCTT |
| | | TCAATGTCGCATTTGCATGCGAAACTTCTCAC |
| | | AGAAGGCCCATTTGACTGCCCATATTCGTACT |
| | | CATACTGGCGAGAAACCTTTCGCTTGCGATAT |
| | | ATGTGGTCGTAAGTTTGCACGGTCGGACAACC |
| | | TCACACGCCACACTAAGATACACCTGCGGCAG |
| | | AAGGAC |
| | | (SEQ ID NO: 58) |
| ZFP-2 | RPFQCRICMRNFSRSSNLTR | CGACCATTCCAGTGTCGAATCTGCATGCGCAA |
| | HIRTHTGEKPFACDICGKKF | CTTCAGCCGAAGTTCCAACCTGACACGGCATA |
| | ADKRTLIRHTKIHTGSQKPF | TCCGCACCCACACGGGAGAGAAGCCTTTTGCC |
| | QCRICMRNFSQRGNLVRHI | TGCGATATTTGTGGAAAGAAGTTTGCTGACAA |
| | RTHTGEKPFACDICGKKFA | GCGGACCTTAATCCGCCACACCAAGATTCATA |
| | LSFNLTRHTKIHTGSQKPFQ | CTGGGTCCCAGAAACCGTTCCAGTGTAGGATA |
| | CRICMRNFSRSDNLTRHIRT | TGCATGAGGAATTTCTCTCAGCGGGGAAATCT |
| | HTGEKPFACDICGRKFADR | AGTGCGACATATAAGGACGCACACAGGTGAA |
| | SHLARHTKIHLRQKD | AAACCATTTGCATGCGACATCTGTGGCAAAAA |
| | (SEQ ID NO: 59) | GTTTGCGCTGAGCTTCAACTTGACTCGTCACA |
| | | CAAAAATCCATACCGGCAGTCAAAAGCCCTTT |
| | | CAATGTCGCATTTGCATGCGAAACTTCTCACG |
| | | GAGTGACAATCTTACGAGACATATTCGTACTC |
| | | ATACTGGCGAGAAACCTTTCGCTTGCGATATA |
| | | TGTGGTCGTAAGTTTGCAGACCGGAGCCACTT |
| | | AGCCAGGCACACTAAGATACACCTGCGGCAG |
| | | AAGGAC |
| | | (SEQ ID NO: 60) |
| ZFP-3 | RPFQCRICMRNFSDRSALAR | CGACCATTCCAGTGTCGAATCTGCATGCGCAA |
| | HIRTHTGEKPFACDICGKKF | CTTCAGCGACCGGAGCGCGCTGGCACGGCAT |
| | ARSDNLTRHTKIHTGSQKPF | ATCCGCACCCACACGGGAGAGAAGCCTTTTGC |
| | QCRICMRNFSQSGDLTRHIR | CTGCGATATTTGTGGAAAGAAGTTTGCTCGAA |
| | THTGEKPFACDICGKKFAV | GTGACAACTTAACGCGCCACACCAAGATTCAT |
| | RQTLKQHTKIHTGSQKPFQ | ACTGGGTCCCAGAAACCGTTCCAGTGTAGGAT |
| | CRICMRNFSAAGNLTRHIRT | ATGCATGAGGAATTTCTCTCAGTCAGGGGACC |
| | HTGEKPFACDICGRKFARS | TCACTCGTCATATAAGGACGCACACAGGTGAA |
| | DNLTRHTKIHLRQKD | AAACCATTTGCATGCGACATCTGTGGCAAAAA |
| | (SEQ ID NO: 61) | GTTTGCGGTACGACAGACGCTTAAACAACACA |
| | | CAAAAATCCATACCGGCAGTCAAAAGCCCTTT |
| | | CAATGTCGCATTTGCATGCGAAACTTCTCAGC |
| | | CGCTGGTAACTTGACACGACATATTCGTACTC |
| | | ATACTGGCGAGAAACCTTTCGCTTGCGATATA |
| | | TGTGGTCGTAAGTTTGCAAGATCTGATAATCT |
| | | AACGCGTCACACTAAGATACACCTGCGGCAG |
| | | AAGGAC |
| | | (SEQ ID NO: 62) |

### Example 2. ZFPs Increase SCN1A Gene Expression in Human Cells

To examine the ability of ZFP1-ZFP3 to upregulate transcription of SCN1A, the ZFP1-ZFP3 DNA binding domains were fused to a hybrid VP64, p53, and RTA (VPR) tripartite strong transcriptional activator domain to form a chimeric transactivator. The VPR fusion activator domain acts to recruit transcriptional regulatory complexes and increase chromatin accessibility and helps to achieve high levels of gene expression. Thus, the ZFP domain will target the VPR activator to the highly conserved sequence in the proximal promoter region to increase SCN1A gene expression.

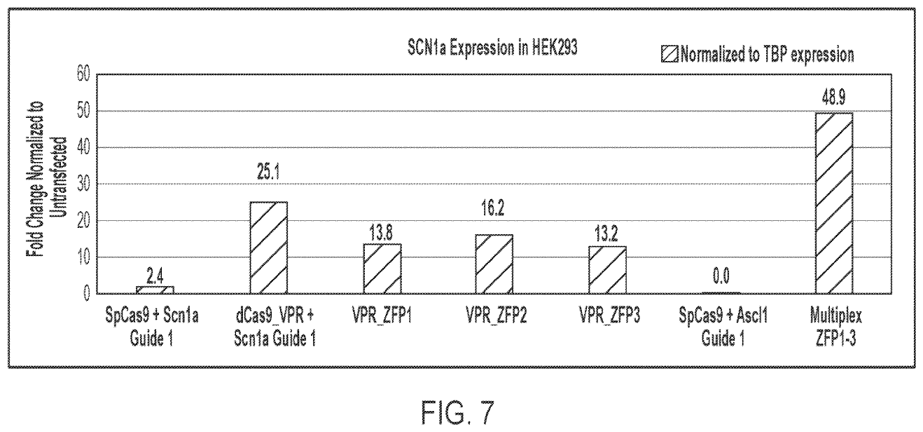

Expression plasmids encoding VPR-ZFP1, VPR-ZFP2, and/or VPR-ZFP3 fusion proteins were transfected via transient transfection into HEK293 cells and SCN1A gene expression was measured by qRT-PCR (using TBP expression as a reference for normalization). The VPR-ZFP fusions comprise ZFP1, ZFP2, and/or ZFP3 fused to VPR. Transfection of three constructs for multiplex regulation, which contained ZFP1, ZFP2, and ZFP3 DNA binding domains each fused to VPR, resulted in 45-fold increased SCN1A gene expression relative to untransfected cells, indicating that the VPR-ZFP chimeric transactivators are able to increase SCN1A gene expression by binding in the promoter proximal region of the gene (FIG. 7).

VPR-[ZFP1-ZFP3] fusion proteins, as well as VPR-ZFP fusion proteins in which the ZFP DNA binding domain is currently being designed, are being transfected in HeLa and HEPG2 cells, both of which have low levels of SCN1A expression. The VPR-ZFP fusion proteins contain single as well as combinations of multiple ZFP DNA binding domains fused to VPR transactivator. SCN1A gene expression is measured by qRT-PCR to determine if these VPR-ZFP fusions are able to increase gene expression. The most promising VPR-ZFP fusion candidates are tested in primary mouse cortical neurons following adeno-associated virus (AAV) delivery of the fusion proteins for the ability to increase SCN1A expression.

The specificity of the ZFP domains is being further optimized using a bacterial one-hybrid selection system (see, e.g., Meng, et al., “Targeted gene inactivation in zebrafish using engineered zinc-finger nucleases,” Nat Biotechnol, 2008) to identify ideal ZFPs from a randomized library in which residues important in DNA binding are varied. The newly-selected ZFPs will be fused to VPR transactivator domains, both individually as well as in combinations of multiple ZFPs and transfected in HEK293, HeLa, and HEPG2 cells, as well as primary mouse cortical neurons to identify the candidate ZFP domains which increase SCN1A gene expression the most following qRT-PCR analysis.

### Example 3. Generate ZFP^{SCN1A} Transactivator Series with Varying Potencies

The most effective ZFPs in upregulating SCN1A gene expression from Example 2 is fused to a series of human transactivation domains (e.g., Rta, p65, Hsf1, etc.) with a gradient of anticipated potencies to identify an assembly that achieves 2-fold upregulation of SCN1A gene expression over a range of AAV multiplicities of infection (MOIs). Mouse primary cortical neurons from normal and SCN1A^{+/-} mice are infected with AAV vectors expressing ZFP^{SCN1A} fusion transactivators. Expression levels of the Nav1.1 protein are assessed by Western blot using and qPCR. Primary neurons treated for 8 hours with TGF-α are used as a positive control because this treatment increases Nav1.1 protein expression by ~6 to 8-fold (Chen et al., 2015, Neuroinflammation 12:126). Changes in the expression levels of other Nav alpha subunit genes are also assessed to demonstrate the specification of ZFP^{SCN1A} transactivation. Immunofluorescence is used to determine whether Nav1.1 expression remains restricted to GABAergic interneurons through double immunofluorescence staining with antibodies to ZFP^{SCN1A} (HA tag) and markers specific for GABAergic neurons (e.g., parvalbumin⁺ or somatostatin⁺) or universal neuronal markers (e.g., NeuN, TUBIII, and/or Map2). The specificity of ZFP^{SCN1A} for transactivation of the SCN1A gene is also assessed by ChIP-Seq and RNA-Seq to map genome binding sites and the resulting transcriptomic prolife generated upon gene transfer.

### Example 4. Histone Organization and Epigenomic Landscape of the SCN1A Promoter in GABAergic Inhibitors to Guide the Design of Promoter Activity Dependent SCN1A-ZFP Transactivators

The ability of ZFPs to bind genomic targets depends upon the accessibility of the target sequence (e.g., presence of a nucleosome-free region). This requirement for DNA accessibility is exploited to design ZFP transactivators that are only functional in a subset of cell types based on the presence of DNA target sequence accessibility. Additional restriction in cell type activity is achieved through the use of tissue-specific promoters for ZFP transactivator expression. Small promoters from pufferfish (Takifugu rubripes) somatostatin and neuropeptide Y genes have been shown to drive highly specific transgene expression in cortical and hippocampal inhibitory interneurons in the context of both AAV vectors and lentiviruses. In some embodiments, the combination of AAV-based transcriptional restriction of SCN1A-specific ZFPs that are sensitive to DNA accessibility results in highly specific up-regulation of Nav1.1 protein expression in inhibitory interneurons throughout the brain. This dual regulatory approach will minimize side effects that may result from the ectopic expression of Nav1.1 protein in cells where it is not normally expressed.

The nucleosome structure and epigenetic landscape of the SCN1A promoter is analyzed in both mouse and human GABAergic inhibitory and glutamergic excitatory neurons. This information is used to design GABAergic inhibitory neuron restricted ZFP transactivators through the targeting of sequences that are only accessible around the SCN1A locus in this cell type.

GABAergic inhibitory neurons from transgenic mice expressing TdTomato under a GAD67 promoter and GFP-positive glutamateric excitatory neurons generated by crossing Emx1-IRES-Cre with ROSA26/stop/EGFP mice are isolated using fluorescence activated cell sorting (FACS). Human GABAergic and excitatory neurons are generated from induced pluripotent stem cells (iPS) cells and confirmed using both immunostaining and RT-PCR for markers specific for these cell types, as well as electrophysiological activity. Accessible genomic regions around the SCN1A promoter in mouse and human neuronal populations are characterized using Assay for Transposase-Accessible Chromatin (ATAC-Seq).

ZFP^{SCN1A} transactivators that recognize sequences accessible only in GABAergic neurons are being designed based on differential chromatin accessibility of genomic regions around the SCN1A promoter in inhibitory and excitatory neurons. A series of candidate ZFP-VPR transactivator fusions is being generated to target different SCN1A accessible regions wherein the binding of the transactivators is expected to potently upregulate Nav1.1 expression in inhibitory regions, as well as reveal any undesired induced expression of Nav1.1 expression in excitatory neurons.

Expression studies are conducted in cultured human iPS-derived neuron and mouse SCN1A^{+/-} primary neurons which model Dravet syndrome to determine if ZFP^{SCN1A} transactivators designed to recognize DNA sequences which are only accessible in inhibitory neurons provide the necessary specificity when expressed from AAV vectors under a pan-neuronal human synapsin-1 or inhibitory interneuron-specific promoter. Nav1.1 expression levels are measured by qRT-PCR, Western blot, and double immunofluorescence with neuronal-type specific markers for inhibitory GABAergic (e.g., GABA⁺, GAD65/67⁺, somatostatin, and/or parvalbumin) and excitatory glutamatergic (e.g., Cux1+, FoxG1, +, GABA_{A} receptors, GABA⁻) neurons. The cell-type specificity of ZFP^{SCN1A} transactivators are being designed to target different sequences in the mouse and human SCN1A promoter as chromatin structure and DNA sequence within syntenic regions differs between species. Controls in these experiments include neuronal cultures infected with similar AAV vectors encoding GFP, ZFPs without transactivation domains, or transactivators without ZFP DNA-binding domains.

MicroRNA (miRNA) binding sites are being incorporated within the 3′ untranslated region (3′ UTR) of the ZFP^{SCN1A} transactivators that are restricted to cell types wherein undesired expression is occurring (e.g., glutamatergic excitatory neurons). This approach was previously utilized to restrict expression of AAV-delivered transgenes (Xie, et al., “MicroRNA-regulated, systematically delivered rAAV9: a step closer to CNS-restricted transgene expression,” Mol. Ther. 2011). Differences in the miRNA expression profile of GABAergic inhibitory neurons and other cell types is being determined by small RNA sequencing.

### Example 5. Evaluate the Potential of AAV-ZFP^{SCN1A} Gene Therapy to Correct Sodium Current Deficits in Patient-Derived iPS-Generated GABAergic Interneurons

A critical step towards the development of a ZFP^{SCN1A} transactivator(s) for Dravet syndrome is to demonstrate that these artificial transactivators have the desired function in human neurons. For this purpose, iPS cells from Dravet patients (n=4-6) and non-Dravet patients (n=4) are being obtained. A non-Dravet genetic background is represented within these cells, obviating the need to artificially manipulate gene expression, and thus iPS cells have emerged as the state-of-the-art cell line for biomedical research. CRISPR-Cas9 genome editing technology is being utilized to create isogenic cell lines by repairing the genetic mutation in SCN1A to the wild-type sequence, or by introducing a Dravet-associated mutation into a normal allele within a control cell line. Isogenic lines thereby eliminate the natural variability that arises from comparing cell lines from different human subjects and are thus valuable for confirming and augmenting disease-specific phenotypes. An established inhibitory neuron differentiation protocol and validation pipeline is being used to differentiate the iPS cell lines into forebrain GABAergic inhibitor interneurons.

Inhibitory neurons derived from Dravet patients exhibit reduced sodium currents and impaired action potential firing as determined by whole cell patch clamp electrophysiology measurements. Similar measurements are being performed to confirm that the Dravet-derived neurons described herein recapitulate these disease-associated phenotypes. Sodium current defects occur in inhibitor, but not excitatory neurons in Dravet patients (Sun et al) and thus only inhibitory neurons are being utilized in the current disclosure. Mutation-induced sodium channel defects in Dravet patient-derived inhibitory neurons can be rescued by ectopic expression of wild-type SCN1A (ref 20). Therefore, the methods described in the current disclosure are suitable for testing the efficacy of the ZFP^{SCN1A} transactivators in restoring wild-type sodium channel function and physiology in the context of Dravet syndrome.

GABAergic inhibitory neuronal cultures are being infected with AAV vectors encoding ZFP^{SCN1A} transactivators under universal neuronal or inhibitory neuron-specific promoters. Changes in Nav1.1 expression levels are being assessed by western blot. The restoration of functional sodium currents in inhibitory neurons is being assessed through whole cell patch clamping of untransfected compared with transfected cells. The binding of ZFP^{SCN1A} transactivators across the genome in all patient-derived inhibitory neurons is being analyzed by ChIP-seq and correlated with any identified transcriptome changes detected by RNA-seq. Controls in these experiments are neuronal cultures infected with similar AAV vectors encoding GFP, ZFPs without VPR transactivator domains, and VPR transactivator domains without ZFP DNA binding domains.

### Example 6. Assessing the Therapeutic Potential of AAV-ZFP^{SCN1A} Intervention at Different Ages and Delivery Routes in SCN1A Mice

The broad tropism of AAV is a critical property for gene therapy applications for broadly expressed genes, but can become a significant challenge when a transgene of interest is expressed in a cell-type specific manner. This has been largely solved for major tissues in the body such as liver, muscle, and heart through the use of tissue specific promoters such as the thyroxin binding protein (TBP), Creatine Kinase and Troponin T, respectively. An additional level of control can be superimposed on tissue specific promoters to achieve a higher degree of de-targeting from specific tissues by incorporation of multiple copies of binding sites for microRNAs highly abundant in those tissues, such as miR-122 in liver and miR-1 in skeletal muscle. The recently described AAV-PHP.B serotype is exceptionally efficient for CNS gene transfer after systemic delivery, where it transduces a broad range of cell types. Moreover its tropism to peripheral tissues is for the most part as broad as that for AAV9. The goal of a gene therapy approach for Dravet syndrome is to restore Nav1.1 expression in GABAergic inhibitory interneurons exclusively while preventing deleterious effects from ectopic expression in other neurons and elsewhere. AAV and lentivirus vectors encoding GFP under small promoters (<2.8 kb) derived from the pufferfish (Takifugu rubripes) somatostatin (fSST), and neuropeptide Y (fNPY) genes have been shown to drive inhibitory neuron specific expression in the mouse brain upon intracranial injection. AAV-PHP.B vectors carrying these promoters driving GFP expression are being compared to control vectors where transgene expression is driven by the ubiquitous strong CAG promoter and the minimal relatively weak mouse MeCP2 promoter. The specificity of AAV-PHP.B-GFP vectors with fSST and fNYP promoters for GABAergic inhibitory interneurons is being studied upon delivery to the CNS by systemic administration in 6 week-old (tail vein) and post-natal day 1 (retro-orbital) mice, CSF delivery in neonates, and lastly unilateral injections targeting the dentate gyrus (DG) (Table 5). The efficiency of CNS gene transfer varies considerably with delivery route and because Scn1a^{+/-} mice at different ages are being treated, a broad analysis is being conducted to establish the baseline of neuronal transduction efficacy and promoter specificity to GABAergic inhibitory interneurons throughout the CNS for each delivery route. AAV vectors driving GFP expression from the short fSST and fNYP promoters have previously been shown to be highly specific for inhibitory interneurons in the hippocampus after direct injection. The AAV-PHP.B vectors of the current disclosure are being validated in the same manner as in subsequent studies, wherein the therapeutic impact of restoring Nav1.1 expression in inhibitory neurons in the hippocampal formation of Scn1a^{+/-} mice, specifically located in the dentate gyrus and the inner lining of the granular cell layer is being assessed (rationale articulated below). Experiments are being conducted in 129SvJ/C57BL/6 mice generated at UMMS by mating 129SvJ with C57BL/6 mice obtained from Jackson Laboratories (Bar Harbor, ME). Mice are being euthanized at one month post-injection and the brain and spinal cord are being collected for histological analysis of transduction efficiency and specificity using double immunofluorescence with antibodies for cell specific markers and GFP. The gene transfer efficiency and specificity for GABAergic inhibitory interneurons is being assessed throughout the brain and spinal cord by double immunofluorescence staining with antibodies to glutamic acid decarboxylase (GAD; marker for GABAergic neurons) and GFP. In addition the preferential specificity of promoters and/or AAV-PHP.B for subsets of inhibitory interneurons expressing somatostatin (SST), parvalbumin (PV), calretinin (CR), vasoactive intestinal peptide (VIP) or neuropeptide Y (NPY) using antibodies specific for those proteins and GFP is assessed. Liver, heart and skeletal muscle are collected from mice treated by systemic and ICV administration to assess GFP expression histologically and western blots are being utilized to determine the possibility of ectopic expression in peripheral tissues.

TABLE 5
Experimental groups

| | Number of mice per cohort | | | |
|---|---|---|---|---|
| | Delivery route | | | |
| | Systemic | | ICV | IC |
| | Age | | | |
| | 6 weeks* | PND1 ^{#} | PND1 ^{#} | 8 weeks* |
| Dose (vg) | 2 × 10¹² | 4 × 10¹¹ | 4 × 10¹⁰ | 1 × 10¹⁰ |
| AAV-fSST-GFP | 6 | 6-8 | 6-8 | 4 |
| AAV-fNYP-GFP | 6 | 6-8 | 6-8 | 4 |
| AAV-CAG-GFP | 6 | 6-8 | 6-8 | 4 |
| AAV-MeCP2-GFP | 6 | 6-8 | 6-8 | — |
| Vehicle (PBS) | 2 | — | — | |

*Groups are composed of equal number of mice from both sexes.
^{#} One litter injected per vector
Abbreviations:
ICV—Intracerebroventricular injection;
IC—Intracranial injection;
PND1—Post-natal day 1

Six week-old Scn1a^{+/-} mice are administered bilateral injections into the dentate gyrus of AAV-PHP.B vectors encoding different ZFP^{Scn1a} transactivator proteins, a construct with the ZFP^{Scn1a} activation domain but without the DNA-binding domain to control for the impact of the activator alone, or the same volume of phosphate buffered saline (PBS) (n=3 males+3 females/group). The single-stranded AAV vectors used in these experiments also carry an IRES-GFP cassette downstream of the ZFP^{Scn1a} cDNA to facilitate identification of transduced cells. At least two ZFP^{Scn1a} transactivators are tested, which may have broader activation in a variety of neurons, as well as the two most promising GABAergic inhibitory neuron restricted ZFP^{SCN1A} transactivators described above. One month post-injection, the brain is harvested and the hippocampus from the one brain hemisphere is dissected to assess expression levels of ZFP^{Scn1a} Nav1.1, Nav1.3, GAD65, GAD67 proteins by western blot using beta-actin or tubulin as loading controls. The other brain hemisphere is examined by histological studies using serial brain sections (10 μm) to analyze % transduced inhibitory interneurons in the dentate gyrus and inner leaflet of the granule cell layer by double immunofluorescence staining with antibodies to GAD and GFP, or GAD and an epitope tag included in all ZFP^{Scn1a} proteins (HA or myc tag). Also, the percentage of GAD-positive neurons that express Nav1.1 and Nav1.3 is being determined to demonstrate restoration of the normal patterns of sodium channel expression. In addition to immunofluorescence detection of Nav1.1 and Nav1.3 protein expression, changes in mRNA levels in GABAergic interneurons are assessed using RNAscope probes for Nav1.1, Nav1.3, ZFP^{Scn1a} and GAD. RNAScope is a highly sensitive in situ hybridization technique to analyze mRNAs levels in neurons in the brain. The combination of these two approaches to assess changes in Nav1.1 levels resulting from ZFP^{Scn1a} expression provides a comprehensive understanding of how changes in interneurons are being achieved by the gene therapy approach of the current disclosure.

The therapeutic efficacy of AAV-PHP.B-ZFP^{Scn1a} gene therapy is analyzed in Scn1a^{+/-} mice of both sexes initiated at post-natal day 1, or 6 weeks of age via the tail vein. Controls include mice treated with an AAV vector encoding a ZFP-like protein without the ZFP DNA-binding domain, as well as age matched untreated Scn1a^{+/-} mice and wild type littermates (n=15 males and 15 females per group). A subset of mice in each group (n=3 males and 3 females) is being euthanized at 12 weeks of age to assess gene transfer efficiency to GABAergic interneurons using western blot as well as immunofluorescence with antibodies to GAD (and other neuronal type specific markers, e.g., GAD65, GAD67) and the ZFP, and restoration of Nav1.1 expression in those cells throughout the brain and spinal cord. Moreover, ectopic expression of ZFP is assessed, along with Nav1.1 expression in peripheral tissues. The other subset of animals in each group (n=24) is being used to study impact on survival (up to 1 year of age), motor performance and behavior, which is being tested every two months from 2-12 months of age. Motor function and coordination is assessed using the accelerating rotarod and beam crossing tests, as Scn1a^{+/-} mice display impaired coordination of forelimbs and hindlimbs by PND21. In addition, behavior tests are being utilized in which Scn1a^{+/-} mice show impaired performance, including: open field, elevated plus maze, nest building, marble burying, and Barnes maze to test spatial learning and memory that appears to be severely compromised in Scn1a^{+/-} mice. The spontaneous seizures characteristic of Dravet syndrome patients are also apparent in Scn1a^{+/-} mice and the frequency increases with age and body temperature. Moreover premature sudden death of Scn1a^{+/-} mice occurs immediately after tonic-clonic seizures. Therefore continuous video monitoring is being utilized for 24 hrs at 2, 6 and 12 months of age to assess seizure frequency and duration. Social interaction studies using chamber preference readouts in response to new objects, smells and mice is being considered if a significant change is detected in primary outcomes measured in the tests described above. Brain, spinal cord and peripheral organs are being collected and assessed at the experimental for humane endpoints to perform the molecular and histological analyses outlined above.

### Example 7. ZFPs and dCas9 Systems Increase SCN1A Gene Expression in Human Cells

To examine the ability of ZFP1-ZFP3 to upregulate transcription of SCN1A, the ZFP1-ZFP3 DNA binding domains were fused to a hybrid VP64, p53, and RTA (VPR) tripartite strong transcriptional activator domain to form a chimeric transactivator. The VPR fusion activator domain acts to recruit transcriptional regulatory complexes and increase chromatin accessibility and helps to achieve high levels of gene expression. Thus, the ZFP domain will target the VPR activator to the highly conserved sequence in the proximal promoter region to increase SCN1A gene expression.

Further, to examine the ability of dCas9 systems that target SCN1A to upregulate transcription of SCN1A, three guide RNAs targeting SCN1A were complexed with dCas9 protein.

HEK293T cells were transiently transfected with one of the following experimental conditions—(1) a VPR-ZFP1 construct; (2) a VPR-ZFP2 construct; (3) a VPR-ZFP3 construct; (4) all three of the VPR-ZFP1, VPR-ZFP2, and VPR-ZFP3 constructs; (5) a dCas9-VPR construct and SCN1A guide RNA 1; (6) a dCas9-VPR construct and SCN1A guide RNA 2; (7) a dCas9-VPR construct and SCN1A guide RNA 3; (8) a dCas9-VPR construct and all three of SCN1A guide RNA 1, SCN1A guide RNA 2, and SCN1A guide RNA 3; and (9) a dCas9-VPR construct without any guide RNA (control). SCN1A gene expression was measured by qRT-PCR. Fold activation of SCN1A was normalized to the control experiment (dCas9-VPR construct without any guide RNA).

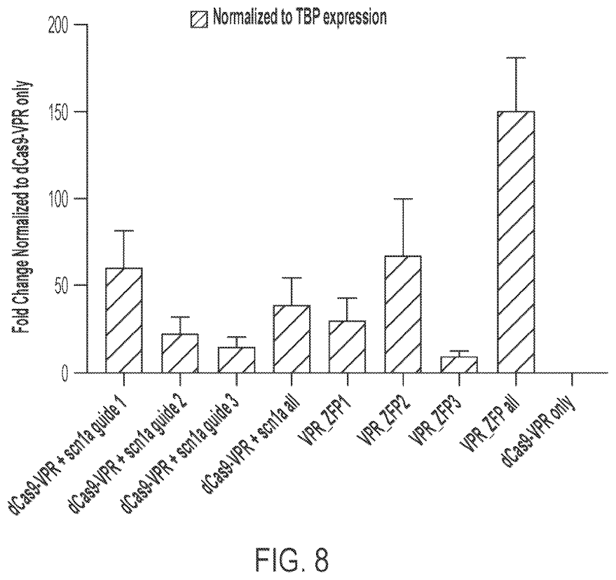

All tested experimental conditions produced increases in gene activation of SCN1A relative to the control experiment (FIG. 8). These data demonstrate that the zinc finger proteins described in this Example and throughout the present disclosure are capable of targeting SCN1A to influence gene expression. These data further demonstrate that the guide RNA sequences of this Example (SEQ ID NOs: 83-94) are capable of targeting dCas9 to SCN1A in order to influence gene expression.

TABLE 6
Guide nucleic acids that target SCN1A (spacer sequence in bold)

| | Nucleotide sequence (DNA) | Nucleotide sequence (RNA) |
|---|---|---|
| SCN1A guide 1 | GAGGTACCATAGAGTGAGGCG | GAGGUACCAUAGAGUGAGGCGGUU |
| | GTTTTAGAGCTAGAAATAGCAA | UUAGAGCUAGAAAUAGCAAGUUAA |
| | GTTAAAATAAGGCTAGTCCGTTA | AAUAAGGCUAGUCCGUUAUCAACUU |
| | TCAACTTGAAAAAGTGGCACCG | GAAAAAGUGGCACCGAGUCGGUGC |
| | AGTCGGTGC (SEQ ID NO: 83) | (SEQ ID NO: 84) |
| SCN1A guide 2 | ACCGAGGCGAGGATGAAGCCG | ACCGAGGCGAGGAUGAAGCCGAGG |
| | AGGTTTTAGAGCTAGAAATAGC | UUUUAGAGCUAGAAAUAGCAAGUU |
| | AAGTTAAAATAAGGCTAGTCCGT | AAAAUAAGGCUAGUCCGUUAUCAAC |
| | TATCAACTTGAAAAAGTGGCACC | UUGAAAAAGUGGCACCGAGUCGGUG |
| | GAGTCGGTGC (SEQ ID NO: 87) | C (SEQ ID NO: 88) |
| SCN1A guide 3 | ACCGAAGCCGAGAGGATACTG | ACCGAAGCCGAGAGGAUACUGCAG |
| | CAGGTTTTAGAGCTAGAAATAG | GUUUUAGAGCUAGAAAUAGCAAGU |
| | CAAGTTAAAATAAGGCTAGTCC | UAAAAUAAGGCUAGUCCGUUAUCAA |
| | GTTATCAACTTGAAAAAGTGGCA | CUUGAAAAAGUGGCACCGAGUCGGU |
| | CCGAGTCGGTGC (SEQ ID NO: 91) | GC (SEQ ID NO: 92) |

1-37. (canceled)

38. The method of any claim 67, wherein the at least one DNA binding domain encodes a zinc finger protein (ZFP), a transcription-activator like effectors (TALEs), a dCas protein (e.g., dCas9 or dCas12a), and/or a homeodomain.

39. (canceled)

40. The method of claim 67, wherein the at least one DNA binding domain is a zinc finger protein comprising a recognition helix encoded by a nucleic acid having a sequence set forth in any one of SEQ ID NOs: 11-16, 23-28, or 35-40.

41. The method of claim 40, wherein the at least one DNA binding domain is a zinc finger protein comprising a recognition helix encoded by a nucleic acid comprising SEQ ID NO: 11, a recognition helix encoded by a nucleic acid comprising SEQ ID NO: 12, a recognition helix encoded by a nucleic acid comprising SEQ ID NO: 13, a recognition helix encoded by a nucleic acid comprising SEQ ID NO: 14, a recognition helix encoded by a nucleic acid comprising SEQ ID NO: 15, and/or a recognition helix encoded by a nucleic acid comprising SEQ ID NO: 16.

42. The method of claim 40, wherein the at least one DNA binding domain is a zinc finger protein comprising a recognition helix encoded by a nucleic acid comprising SEQ ID NO: 23, a recognition helix encoded by a nucleic acid comprising SEQ ID NO: 24, a recognition helix encoded by a nucleic acid comprising SEQ ID NO: 25, a recognition helix encoded by a nucleic acid comprising SEQ ID NO: 26, a recognition helix encoded by a nucleic acid comprising SEQ ID NO: 27, and/or a recognition helix encoded by a nucleic acid comprising SEQ ID NO: 28.

43. The method of claim 40, wherein the at least one DNA binding domain is a zinc finger protein comprising a recognition helix encoded by a nucleic acid comprising SEQ ID NO: 35, a recognition helix encoded by a nucleic acid comprising SEQ ID NO: 36, a recognition helix encoded by a nucleic acid comprising SEQ ID NO: 37, a recognition helix encoded by a nucleic acid comprising SEQ ID NO: 38, a recognition helix encoded by a nucleic acid comprising SEQ ID NO: 39, and/or a recognition helix encoded by a nucleic acid comprising SEQ ID NO: 40.

44. The method of claim 67, wherein the at least one DNA binding domain is a zinc finger protein comprising the sequence set forth in any one of SEQ ID NOs: 17-22, 29-34, or 41-46.

45. The method of claim 44, wherein the at least one DNA binding domain is a zinc finger protein comprising a recognition helix comprising SEQ ID NO: 17, a recognition helix comprising SEQ ID NO: 18, a recognition helix comprising SEQ ID NO: 19, a recognition helix comprising SEQ ID NO: 20, a recognition helix comprising SEQ ID NO: 21, and/or a recognition helix comprising SEQ ID NO: 22.

46. The method of claim 44, wherein the at least one DNA binding domain is a zinc finger protein comprising a recognition helix comprising SEQ ID NO: 29, a recognition helix comprising SEQ ID NO: 30, a recognition helix comprising SEQ ID NO: 31, a recognition helix comprising SEQ ID NO: 32, a recognition helix comprising SEQ ID NO: 33, and/or a recognition helix comprising SEQ ID NO: 34.

47. The method of claim 44, wherein the at least one DNA binding domain is a zinc finger protein comprising a recognition helix comprising SEQ ID NO: 41, a recognition helix comprising SEQ ID NO: 42, a recognition helix comprising SEQ ID NO: 43, a recognition helix comprising SEQ ID NO: 44, a recognition helix comprising SEQ ID NO: 45, and/or a recognition helix comprising SEQ ID NO: 46.

48-51. (canceled)

52. The method of claim 67, wherein the at least one transcriptional regulator domain is a transactivator derived from VRP, Rta, p65, Hsf1 or any combination thereof.

53. The method of claim 67, wherein the transgene encoding the at least one transcriptional regulator transgene comprises the sequence set forth in SEQ ID NO: 47.

54. The method of claim 67, wherein the at least one transcriptional regulator domain is encoded by the amino acid sequence set forth in SEQ ID NO: 48.

55-58. (canceled)

59. The rAAV of any one of claims 32-58, wherein the AAV capsid serotype is selected from the group consisting of AAV1, AAV2, AAV3, AAV4, AAV5, AAV6, AAV7, AAV8, AAVrh8, AAV9, AAV10, AAVrh10, or AAV.PHPB.

60-63. (canceled)

64. The method of claim 67, wherein the transgene is operably linked to a promoter.

65. The method of claim 64, wherein the promoter is a tissue-specific promoter.

66. (canceled)

67. A method of increasing expression of a target gene, the method comprising administering to a cell or subject comprising the target gene a recombinant adeno-associated virus (rAAV) comprising:
(i) a nucleic acid comprising a transgene encoding at least one DNA binding domain fused to at least one transcriptional regulator domain, wherein the DNA binding domain binds to a target gene or a regulatory region of a target gene wherein the target gene encodes a voltage-gated sodium channel,
(ii) at least one capsid protein.

68. The method of claim 67, wherein the subject is haploinsufficient for the target gene.

69. The method of claim 67, wherein the target gene is SCN1A.

70-71. (canceled)

72. A method of treating Dravet syndrome in a subject, comprising administering to a subject expressing a target gene a recombinant adeno-associated virus (rAAV) comprising:
(i) a nucleic acid comprising a transgene encoding at least one DNA binding domain fused to at least one transcriptional regulator domain, wherein the DNA binding domain binds to a target gene or a regulatory region of a target gene wherein the target gene encodes a voltage-gated sodium channel,
(ii) at least one capsid protein.

73-76. (canceled)

77. The method of claim 72, wherein the isolated nucleic acid or the rAAV is administered by intravenous injection, intramuscular injection, inhalation, subcutaneous injection, and/or intracranial injection.

78-88. (canceled)


SEQUENCE LISTING (121 sequences)

SEQ ID NO: 1 - DNA, 672 bp, Homo sapiens
  source 1..672: mol_type = genomic DNA
aatttccatggactctttttccaaaggaataactggaatgaataaacttaaaatcaagatgaaacaattagatggcttacctgattaaaaggaaaattatccatctgcagtgaggaacagcatcacccaaagacgagatgataacaatgtgccttcagttgcaattgttcagttccttcttgcaaaaggtgtcaaagtatttacaagggctgcagtctcactggggcagaacacacagacacacaaacacacacaaacgcacacatacacacatgcaccagagacctctgcagtatcctctcggcttcatcctcgcctcactctatggtacctaatacaaatcagcaaatagcttgtttcaaaaaaaaaaaaaagtcaagacagcaccttacattacatcgccatctagtggctaaatattaaacactttctcacaatccagatttatgatttcttcctcaacctcttttctctcagcttttttcctttcttctctgtaatctcccagtattgcttctccttgcttctctttcattccctattgctatataatatcatgaacctaatgactcaaagaggaaaaggtttgaaagtaaatatagctattttcaagtagtacttgaaaaacttagcattattttagtttgaaactgttactttattcctaatatg

SEQ ID NO: 2 - DNA, 669 bp, Mus musculus
  source 1..669: mol_type = genomic DNA
tatttccgtgggctcttctccccaaggatttaccaggtaagaattcaccaccaaagaagatcacaatgagataatcagatggcttacctgataaaaaggaaaattatccatctgcagtcaggagcaacatctccccacgacgagtccgcaccttccgttgcaacgattcagattccttcttgcaaaaggtgaccaagtgcttcacaagggctgcagcctcataggggagaacacacgtacacaaacacacgcacacacacacacacatgcaccagagacctctgcagtatcctctggcttcatcctcgcctcactctatggtacctaatacaaatcagcaaatagcttgttttaaaaaaaagaaagaaaaaaagcggagacagcacctaacgttacagtgccatctagtggctacatcgtaaataggttctcacagcctggatttctgtgttctttctcaaccgcttccttctggttcctttttcttttttcctctttattttggttttattacttcctcagatgcctttttttcattcccctttgctctgcctacatggaactattgacttaaagattaaaacaatcagaactggagagcgttgcttttaagttaaaaaaaaaaaggttgctaattttgtttgtaaatgttactttattttctctatt

SEQ ID NO: 3 - DNA, 130 bp, synthetic construct
  misc_feature 1..130: note = Synthetic
  source 1..130: mol_type = other DNA
tttttttttttttttttgaaacaagctatttgctgatttgtattaggtaccatagagtgaggcgaggatgaagccgagaggatactgcagaggtctctggtgcatgtgtgtatgtgtgcgtttgtgtgtg

SEQ ID NO: 4 - DNA, 41 bp, synthetic construct
  misc_feature 1..41: note = Synthetic
  source 1..41: mol_type = other DNA
gagtgaggcgaggatgaagccgagaggatactgcagaggtc

SEQ ID NO: 5 - DNA, 18 bp, synthetic construct
  misc_feature 1..18: note = Synthetic
  source 1..18: mol_type = other DNA
gagtgaggcgaggatgaa

SEQ ID NO: 6 - DNA, 18 bp, synthetic construct
  misc_feature 1..18: note = Synthetic
  source 1..18: mol_type = other DNA
ggcgaggatgaagccgag

SEQ ID NO: 7 - DNA, 18 bp, synthetic construct
  misc_feature 1..18: note = Synthetic
  source 1..18: mol_type = other DNA
gaggatactgcagaggtc

SEQ ID NO: 8 - protein, 5 aa, synthetic construct
  REGION 1..5: note = Synthetic
EGEDE

SEQ ID NO: 9 - protein, 6 aa, synthetic construct
  REGION 1..6: note = Synthetic
GEDEAE

SEQ ID NO: 10 - protein, 6 aa, synthetic construct
  REGION 1..6: note = Synthetic
EDTAEV

SEQ ID NO: 11 - DNA, 21 bp, synthetic construct
  misc_feature 1..21: note = Synthetic
  source 1..21: mol_type = other DNA
cagcggggaaacctggtgagg

SEQ ID NO: 12 - DNA, 21 bp, synthetic construct
  misc_feature 1..21: note = Synthetic
  source 1..21: mol_type = other DNA
ctgagcttcaatctaaccaga

SEQ ID NO: 13 - DNA, 21 bp, synthetic construct
  misc_feature 1..21: note = Synthetic
  source 1..21: mol_type = other DNA
cggagtgacaacttaacgcgg

SEQ ID NO: 14 - DNA, 21 bp, synthetic construct
  misc_feature 1..21: note = Synthetic
  source 1..21: mol_type = other DNA
gaccggtctcaccttgcccga

SEQ ID NO: 15 - DNA, 21 bp, synthetic construct
  misc_feature 1..21: note = Synthetic
  source 1..21: mol_type = other DNA
cagaaggcccatttgactgcc

SEQ ID NO: 16 - DNA, 21 bp, synthetic construct
  misc_feature 1..21: note = Synthetic
  source 1..21: mol_type = other DNA
cggtcggacaacctcacacgc

SEQ ID NO: 17 - protein, 7 aa, synthetic construct
  REGION 1..7: note = Synthetic
QRGNLVR

SEQ ID NO: 18 - protein, 7 aa, synthetic construct
  REGION 1..7: note = Synthetic
LSFNLTR

SEQ ID NO: 19 - protein, 7 aa, synthetic construct
  REGION 1..7: note = Synthetic
RSDNLTR

SEQ ID NO: 20 - protein, 7 aa, synthetic construct
  REGION 1..7: note = Synthetic
DRSHLAR

SEQ ID NO: 21 - protein, 7 aa, synthetic construct
  REGION 1..7: note = Synthetic
QKAHLTA

SEQ ID NO: 22 - protein, 7 aa, synthetic construct
  REGION 1..7: note = Synthetic
RSDNLTR

SEQ ID NO: 23 - DNA, 21 bp, synthetic construct
  misc_feature 1..21: note = Synthetic
  source 1..21: mol_type = other DNA
cgaagttccaacctgacacgg

SEQ ID NO: 24 - DNA, 21 bp, synthetic construct
  misc_feature 1..21: note = Synthetic
  source 1..21: mol_type = other DNA
gacaagcggaccttaatccgc

SEQ ID NO: 25 - DNA, 21 bp, synthetic construct
  misc_feature 1..21: note = Synthetic
  source 1..21: mol_type = other DNA
cagcggggaaatctagtgcga

SEQ ID NO: 26 - DNA, 21 bp, synthetic construct
  misc_feature 1..21: note = Synthetic
  source 1..21: mol_type = other DNA
ctgagcttcaacttgactcgt

SEQ ID NO: 27 - DNA, 21 bp, synthetic construct
  misc_feature 1..21: note = Synthetic
  source 1..21: mol_type = other DNA
cggagtgacaatcttacgaga

SEQ ID NO: 28 - DNA, 21 bp, synthetic construct
  misc_feature 1..21: note = Synthetic
  source 1..21: mol_type = other DNA
gaccggagccacttagccagg

SEQ ID NO: 29 - protein, 7 aa, synthetic construct
  REGION 1..7: note = Synthetic
RSSNLTR

SEQ ID NO: 30 - protein, 7 aa, synthetic construct
  REGION 1..7: note = Synthetic
DKRTLIR

SEQ ID NO: 31 - protein, 7 aa, synthetic construct
  REGION 1..7: note = Synthetic
QRGNLVR

SEQ ID NO: 32 - protein, 7 aa, synthetic construct
  REGION 1..7: note = Synthetic
LSFNLTR

SEQ ID NO: 33 - protein, 7 aa, synthetic construct
  REGION 1..7: note = Synthetic
RSDNLTR

SEQ ID NO: 34 - protein, 7 aa, synthetic construct
  REGION 1..7: note = Synthetic
DRSHLAR

SEQ ID NO: 35 - DNA, 21 bp, synthetic construct
  misc_feature 1..21: note = Synthetic
  source 1..21: mol_type = other DNA
gaccggagcgcgctggcacgg

SEQ ID NO: 36 - DNA, 21 bp, synthetic construct
  misc_feature 1..21: note = Synthetic
  source 1..21: mol_type = other DNA
cgaagtgacaacttaacgcgc

SEQ ID NO: 37 - DNA, 21 bp, synthetic construct
  misc_feature 1..21: note = Synthetic
  source 1..21: mol_type = other DNA
cagtcaggggacctcactcgt

SEQ ID NO: 38 - DNA, 21 bp, synthetic construct
  misc_feature 1..21: note = Synthetic
  source 1..21: mol_type = other DNA
gtacgacagacgcttaaacaa

SEQ ID NO: 39 - DNA, 21 bp, synthetic construct
  misc_feature 1..21: note = Synthetic
  source 1..21: mol_type = other DNA
gccgctggtaacttgacacga

SEQ ID NO: 40 - DNA, 21 bp, synthetic construct
  misc_feature 1..21: note = Synthetic
  source 1..21: mol_type = other DNA
agatctgataatctaacgcgt

SEQ ID NO: 41 - protein, 7 aa, synthetic construct
  REGION 1..7: note = Synthetic
DRSALAR

SEQ ID NO: 42 - protein, 7 aa, synthetic construct
  REGION 1..7: note = Synthetic
RSDNLTR

SEQ ID NO: 43 - protein, 7 aa, synthetic construct
  REGION 1..7: note = Synthetic
QSGDLTR

SEQ ID NO: 44 - protein, 7 aa, synthetic construct
  REGION 1..7: note = Synthetic
VRQTLKQ

SEQ ID NO: 45 - protein, 7 aa, synthetic construct
  REGION 1..7: note = Synthetic
AAGNLTR

SEQ ID NO: 46 - protein, 7 aa, synthetic construct
  REGION 1..7: note = Synthetic
RSDNLTR

SEQ ID NO: 47 - DNA, 1569 bp, synthetic construct
  misc_feature 1..1569: note = Synthetic
  source 1..1569: mol_type = other DNA
gaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 48 - protein, 523 aa, synthetic construct
  REGION 1..523: note = Synthetic
EASGSGRADALDDFDLDMLGSDALDDFDLDMLGSDALDDFDLDMLGSDALDDFDLDMLINSRSSGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 49 - DNA, 6027 bp, Homo sapiens
  source 1..6027: mol_type = genomic DNA
atggaacagaccgtgctggtgccgccgggcccggatagctttaacttttttacccgcgaaagcctggcggcgattgaacgccgcattgcggaagaaaaagcgaaaaacccgaaaccggataaaaaagatgatgatgaaaacggcccgaaaccgaacagcgatctggaagcgggcaaaaacctgccgtttatttatggcgatattccgccggaaatggtgagcgaaccgctggaagatctggatccgtattatattaacaaaaaaacctttattgtgctgaacaaaggcaaagcgatttttcgctttagcgcgaccagcgcgctgtatattctgaccccgtttaacccgctgcgcaaaattgcgattaaaattctggtgcatagcctgtttagcatgctgattatgtgcaccattctgaccaactgcgtgtttatgaccatgagcaacccgccggattggaccaaaaacgtggaatatacctttaccggcatttatacctttgaaagcctgattaaaattattgcgcgcggcttttgcctggaagattttacctttctgcgcgatccgtggaactggctggattttaccgtgattacctttgcgtatgtgaccgaatttgtggatctgggcaacgtgagcgcgctgcgcacctttcgcgtgctgcgcgcgctgaaaaccattagcgtgattccgggcctgaaaaccattgtgggcgcgctgattcagagcgtgaaaaaactgagcgatgtgatgattctgaccgtgttttgcctgagcgtgtttgcgctgattggcctgcagctgtttatgggcaacctgcgcaacaaatgcattcagtggccgccgaccaacgcgagcctggaagaacatagcattgaaaaaaacattaccgtgaactataacggcaccctgattaacgaaaccgtgtttgaatttgattggaaaagctatattcaggatagccgctatcattattttctggaaggctttctggatgcgctgctgtgcggcaacagcagcgatgcgggccagtgcccggaaggctatatgtgcgtgaaagcgggccgcaacccgaactatggctataccagctttgatacctttagctgggcgtttctgagcctgtttcgcctgatgacccaggatttttgggaaaacctgtatcagctgaccctgcgcgcggcgggcaaaacctatatgattttttttgtgctggtgatttttctgggcagcttttatctgattaacctgattctggcggtggtggcgatggcgtatgaagaacagaaccaggcgaccctggaagaagcggaacagaaagaagcggaatttcagcagatgattgaacagctgaaaaaacagcaggaagcggcgcagcaggcggcgaccgcgaccgcgagcgaacatagccgcgaaccgagcgcggcgggccgcctgagcgatagcagcagcgaagcgagcaaactgagcagcaaaagcgcgaaagaacgccgcaaccgccgcaaaaaacgcaaacagaaagaacagagcggcggcgaagaaaaagatgaagatgaatttcagaaaagcgaaagcgaagatagcattcgccgcaaaggctttcgctttagcattgaaggcaaccgcctgacctatgaaaaacgctatagcagcccgcatcagagcctgctgagcattcgcggcagcctgtttagcccgcgccgcaacagccgcaccagcctgtttagctttcgcggccgcgcgaaagatgtgggcagcgaaaacgattttgcggatgatgaacatagcacctttgaagataacgaaagccgccgcgatagcctgtttgtgccgcgccgccatggcgaacgccgcaacagcaacctgagccagaccagccgcagcagccgcatgctggcggtgtttccggcgaacggcaaaatgcatagcaccgtggattgcaacggcgtggtgagcctggtgggcggcccgagcgtgccgaccagcccggtgggccagctgctgccggaagtgattattgataaaccggcgaccgatgataacggcaccaccaccgaaaccgaaatgcgcaaacgccgcagcagcagctttcatgtgagcatggattttctggaagatccgagccagcgccagcgcgcgatgagcattgcgagcattctgaccaacaccgtggaagaactggaagaaagccgccagaaatgcccgccgtgctggtataaatttagcaacatttttctgatttgggattgcagcccgtattggctgaaagtgaaacatgtggtgaacctggtggtgatggatccgtttgtggatctggcgattaccatttgcattgtgctgaacaccctgtttatggcgatggaacattatccgatgaccgatcattttaacaacgtgctgaccgtgggcaacctggtgtttaccggcatttttaccgcggaaatgtttctgaaaattattgcgatggatccgtattattattttcaggaaggctggaacatttttgatggctttattgtgaccctgagcctggtggaactgggcctggcgaacgtggaaggcctgagcgtgctgcgcagctttcgcctgctgcgcgtgtttaaactggcgaaaagctggccgaccctgaacatgctgattaaaattattggcaacagcgtgggcgcgctgggcaacctgaccctggtgctggcgattattgtgtttatttttgcggtggtgggcatgcagctgtttggcaaaagctataaagattgcgtgtgcaaaattgcgagcgattgccagctgccgcgctggcatatgaacgatttttttcatagctttctgattgtgtttcgcgtgctgtgcggcgaatggattgaaaccatgtgggattgcatggaagtggcgggccaggcgatgtgcctgaccgtgtttatgatggtgatggtgattggcaacctggtggtgctgaacctgtttctggcgctgctgctgagcagctttagcgcggataacctggcggcgaccgatgatgataacgaaatgaacaacctgcagattgcggtggatcgcatgcataaaggcgtggcgtatgtgaaacgcaaaatttatgaatttattcagcagagctttattcgcaaacagaaaattctggatgaaattaaaccgctggatgatctgaacaacaaaaaagatagctgcatgagcaaccataccgcggaaattggcaaagatctggattatctgaaagatgtgaacggcaccaccagcggcattggcaccggcagcagcgtggaaaaatatattattgatgaaagcgattatatgagctttattaacaacccgagcctgaccgtgaccgtgccgattgcggtgggcgaaagcgattttgaaaacctgaacaccgaagattttagcagcgaaagcgatctggaagaaagcaaagaaaaactgaacgaaagcagcagcagcagcgaaggcagcaccgtggatattggcgcgccggtggaagaacagccggtggtggaaccggaagaaaccctggaaccggaagcgtgctttaccgaaggctgcgtgcagcgctttaaatgctgccagattaacgtggaagaaggccgcggcaaacagtggtggaacctgcgccgcacctgctttcgcattgtggaacataactggtttgaaacctttattgtgtttatgattctgctgagcagcggcgcgctggcgtttgaagatatttatattgatcagcgcaaaaccattaaaaccatgctggaatatgcggataaagtgtttacctatatttttattctggaaatgctgctgaaatgggtggcgtatggctatcagacctattttaccaacgcgtggtgctggctggattttctgattgtggatgtgagcctggtgagcctgaccgcgaacgcgctgggctatagcgaactgggcgcgattaaaagcctgcgcaccctgcgcgcgctgcgcccgctgcgcgcgctgagccgctttgaaggcatgcgcgtggtggtgaacgcgctgctgggcgcgattccgagcattatgaacgtgctgctggtgtgcctgattttttggctgatttttagcattatgggcgtgaacctgtttgcgggcaaattttatcattgcattaacaccaccaccggcgatcgctttgatattgaagatgtgaacaaccataccgattgcctgaaactgattgaacgcaacgaaaccgcgcgctggaaaaacgtgaaagtgaactttgataacgtgggctttggctatctgagcctgctgcaggtggcgacctttaaaggctggatggatattatgtatgcggcggtggatagccgcaacgtggaactgcagccgaaatatgaagaaagcctgtatatgtatctgtattttgtgatttttattatttttggcagcttttttaccctgaacctgtttattggcgtgattattgataactttaaccagcagaaaaaaaaatttggcggccaggatatttttatgaccgaagaacagaaaaaatattataacgcgatgaaaaaactgggcagcaaaaaaccgcagaaaccgattccgcgcccgggcaacaaatttcagggcatggtgtttgattttgtgacccgccaggtgtttgatattagcattatgattctgatttgcctgaacatggtgaccatgatggtggaaaccgatgatcagagcgaatatgtgaccaccattctgagccgcattaacctggtgtttattgtgctgtttaccggcgaatgcgtgctgaaactgattagcctgcgccattattattttaccattggctggaacatttttgattttgtggtggtgattctgagcattgtgggcatgtttctggcggaactgattgaaaaatattttgtgagcccgaccctgtttcgcgtgattcgcctggcgcgcattggccgcattctgcgcctgattaaaggcgcgaaaggcattcgcaccctgctgtttgcgctgatgatgagcctgccggcgctgtttaacattggcctgctgctgtttctggtgatgtttatttatgcgatttttggcatgagcaactttgcgtatgtgaaacgcgaagtgggcattgatgatatgtttaactttgaaacctttggcaacagcatgatttgcctgtttcagattaccaccagcgcgggctgggatggcctgctggcgccgattctgaacagcaaaccgccggattgcgatccgaacaaagtgaacccgggcagcagcgtgaaaggcgattgcggcaacccgagcgtgggcatttttttttttgtgagctatattattattagctttctggtggtggtgaacatgtatattgcggtgattctggaaaactttagcgtggcgaccgaagaaagcgcggaaccgctgagcgaagatgattttgaaatgttttatgaagtgtgggaaaaatttgatccggatgcgacccagtttatggaatttgaaaaactgagccagtttgcggcggcgctggaaccgccgctgaacctgccgcagccgaacaaactgcagctgattgcgatggatctgccgatggtgagcggcgatcgcattcattgcctggatattctgtttgcgtttaccaaacgcgtgctgggcgaaagcggcgaaatggatgcgctgcgcattcagatggaagaacgctttatggcgagcaacccgagcaaagtgagctatcagccgattaccaccaccctgaaacgcaaacaggaagaagtgagcgcggtgattattcagcgcgcgtatcgccgccatctgctgaaacgcaccgtgaaacaggcgagctttacctataacaaaaacaaaattaaaggcggcgcgaacctgctgattaaagaagatatgattattgatcgcattaacgaaaacagcattaccgaaaaaaccgatctgaccatgagcaccgcggcgtgcccgccgagctatgatcgcgtgaccaaaccgattgtggaaaaacatgaacaggaaggcaaagatgaaaaagcgaaaggcaaa

SEQ ID NO: 50 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 51 - DNA, 1470 bp, Homo sapiens
  source 1..1470: mol_type = genomic DNA
atggatctgctggtggatgaactgtttgcggatatgaacgcggatggcgcgagcccgccgccgccgcgcccggcgggcggcccgaaaaacaccccggcggcgccgccgctgtatgcgaccggccgcctgagccaggcgcagctgatgccgagcccgccgatgccggtgccgccggcggcgctgtttaaccgcctgctggatgatctgggctttagcgcgggcccggcgctgtgcaccatgctggatacctggaacgaagatctgtttagcgcgctgccgaccaacgcggatctgtatcgcgaatgcaaatttctgagcaccctgccgagcgatgtggtggaatggggcgatgcgtatgtgccggaacgcacccagattgatattcgcgcgcatggcgatgtggcgtttccgaccctgccggcgacccgcgatggcctgggcctgtattatgaagcgctgagccgcttttttcatgcggaactgcgcgcgcgcgaagaaagctatcgcaccgtgctggcgaacttttgcagcgcgctgtatcgctatctgcgcgcgagcgtgcgccagctgcatcgccaggcgcatatgcgcggccgcgatcgcgatctgggcgaaatgctgcgcgcgaccattgcggatcgctattatcgcgaaaccgcgcgcctggcgcgcgtgctgtttctgcatctgtatctgtttctgacccgcgaaattctgtgggcggcgtatgcggaacagatgatgcgcccggatctgtttgattgcctgtgctgcgatctggaaagctggcgccagctggcgggcctgtttcagccgtttatgtttgtgaacggcgcgctgaccgtgcgcggcgtgccgattgaagcgcgccgcctgcgcgaactgaaccatattcgcgaacatctgaacctgccgctggtgcgcagcgcggcgaccgaagaaccgggcgcgccgctgaccaccccgccgaccctgcatggcaaccaggcgcgcgcgagcggctattttatggtgctgattcgcgcgaaactggatagctatagcagctttaccaccagcccgagcgaagcggtgatgcgcgaacatgcgtatagccgcgcgcgcaccaaaaacaactatggcagcaccattgaaggcctgctggatctgccggatgatgatgcgccggaagaagcgggcctggcggcgccgcgcctgagctttctgccggcgggccatacccgccgcctgagcaccgcgccgccgaccgatgtgagcctgggcgatgaactgcatctggatggcgaagatgtggcgatggcgcatgcggatgcgctggatgattttgatctggatatgctgggcgatggcgatagcccgggcccgggctttaccccgcatgatagcgcgccgtatggcgcgctggatatggcggattttgaatttgaacagatgtttaccgatgcgctgggcattgatgaatatggcggc

SEQ ID NO: 52 - protein, 490 aa, Homo sapiens
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 53 - DNA, 2570 bp, Homo sapiens
  source 1..2570: mol_type = genomic DNA
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaa

SEQ ID NO: 54 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 55 - DNA, 1815 bp, Homo sapiens
  source 1..1815: mol_type = genomic DNA
atgcgcccgaaaaaagatggcctggaagattttctgcgcctgaccccggaaattaaaaaacagctgggcagcctggtgagcgattattgcaacgtgctgaacaaagaatttaccgcgggcagcgtggaaattaccctgcgcagctataaaatttgcaaagcgtttattaacgaagcgaaagcgcatggccgcgaatggggcggcctgatggcgaccctgaacatttgcaacttttgggcgattctgcgcaacaaccgcgtgcgccgccgcgcggaaaacgcgggcaacgatgcgtgcagcattgcgtgcccgattgtgatgcgctatgtgctggatcatctgattgtggtgaccgatcgcttttttattcaggcgccgagcaaccgcgtgatgattccggcgaccattggcaccgcgatgtataaactgctgaaacatagccgcgtgcgcgcgtatacctatagcaaagtgctgggcgtggatcgcgcggcgattatggcgagcggcaaacaggtggtggaacatctgaaccgcatggaaaaagaaggcctgctgagcagcaaatttaaagcgttttgcaaatgggtgtttacctatccggtgctggaagaaatgtttcagaccatggtgagcagcaaaaccggccatctgaccgatgatgtgaaagatgtgcgcgcgctgattaaaaccctgccgcgcgcgagctatagcagccatgcgggccagcgcagctatgtgagcggcgtgctgccggcgtgcctgctgagcaccaaaagcaaagcggtggaaaccccgattctggtgagcggcgcggatcgcatggatgaagaactgatgggcaacgatggcggcgcgagccataccgaagcgcgctatagcgaaagcggccagtttcatgcgtttaccgatgaactggaaagcctgccgagcccgaccatgccgctgaaaccgggcgcgcagagcgcggattgcggcgatagcagcagcagcagcagcgatagcggcaacagcgataccgaacagagcgaacgcgaagaagcgcgcgcggaagcgccgcgcctgcgcgcgccgaaaagccgccgcaccagccgcccgaaccgcggccagaccccgtgcccgagcaacgcggcggaaccggaacagccgtggattgcggcggtgcatcaggaaagcgatgaacgcccgatttttccgcatccgagcaaaccgacctttctgccgccggtgaaacgcaaaaaaggcctgcgcgatagccgcgaaggcatgtttctgccgaaaccggaagcgggcagcgcgattagcgatgtgtttgaaggccgcgaagtgtgccagccgaaacgcattcgcccgtttcatccgccgggcagcccgtgggcgaaccgcccgctgccggcgagcctggcgccgaccccgaccggcccggtgcatgaaccggtgggcagcctgaccccggcgccggtgccgcagccgctggatccggcgccggcggtgaccccggaagcgagccatctgctggaagatccggatgaagaaaccagccaggcggtgaaagcgctgcgcgaaatggcggataccgtgattccgcagaaagaagaagcggcgatttgcggccagatggatctgagccatccgccgccgcgcggccatctggatgaactgaccaccaccctggaaagcatgaccgaagatctgaacctggatagcccgctgaccccggaactgaacgaaattctggatacctttctgaacgatgaatgcctgctgcatgcgatgcatattagcaccggcctgagcatttttgataccagcctgttt

SEQ ID NO: 56 - protein, 605 aa, synthetic construct
  REGION 1..605: note = Synthetic
MRPKKDGLEDFLRLTPEIKKQLGSLVSDYCNVLNKEFTAGSVEITLRSYKICKAFINEAKAHGREWGGLMATLNICNFWAILRNNRVRRRAENAGNDACSIACPIVMRYVLDHLIVVTDRFFIQAPSNRVMIPATIGTAMYKLLKHSRVRAYTYSKVLGVDRAAIMASGKQVVEHLNRMEKEGLLSSKFKAFCKWVFTYPVLEEMFQTMVSSKTGHLTDDVKDVRALIKTLPRASYSSHAGQRSYVSGVLPACLLSTKSKAVETPILVSGADRMDEELMGNDGGASHTEARYSESGQFHAFTDELESLPSPTMPLKPGAQSADCGDSSSSSSDSGNSDTEQSEREEARAEAPRLRAPKSRRTSRPNRGQTPCPSNAAEPEQPWIAAVHQESDERPIFPHPSKPTFLPPVKRKKGLRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 57 - protein, 172 aa, synthetic construct
  REGION 1..172: note = Synthetic
RPFQCRICMRNFSQRGNLVRHIRTHTGEKPFACDICGKKFALSFNLTRHTKIHTGSQKPFQCRICMRNFSRSDNLTRHIRTHTGEKPFACDICGKKFADRSHLARHTKIHTGSQKPFQCRICMRNFSQKAHLTAHIRTHTGEKPFACDICGRKFARSDNLTRHTKIHLRQKD

SEQ ID NO: 58 - DNA, 516 bp, synthetic construct
  misc_feature 1..516: note = Synthetic
  source 1..516: mol_type = other DNA
cgaccattccagtgtcgaatctgcatgcgcaacttcagccagcggggaaacctggtgaggcatatccgcacccacacgggagagaagccttttgcctgcgatatttgtggaaagaagtttgctctgagcttcaatctaaccagacacaccaagattcatactgggtcccagaaaccgttccagtgtaggatatgcatgaggaatttctctcggagtgacaacttaacgcggcatataaggacgcacacaggtgaaaaaccatttgcatgcgacatctgtggcaaaaagtttgcggaccggtctcaccttgcccgacacacaaaaatccataccggcagtcaaaagccctttcaatgtcgcatttgcatgcgaaacttctcacagaaggcccatttgactgcccatattcgtactcatactggcgagaaacctttcgcttgcgatatatgtggtcgtaagtttgcacggtcggacaacctcacacgccacactaagatacacctgcggcagaaggac

SEQ ID NO: 59 - protein, 172 aa, synthetic construct
  REGION 1..172: note = Synthetic
RPFQCRICMRNFSRSSNLTRHIRTHTGEKPFACDICGKKFADKRTLIRHTKIHTGSQKPFQCRICMRNFSQRGNLVRHIRTHTGEKPFACDICGKKFALSFNLTRHTKIHTGSQKPFQCRICMRNFSRSDNLTRHIRTHTGEKPFACDICGRKFADRSHLARHTKIHLRQKD

SEQ ID NO: 60 - DNA, 516 bp, synthetic construct
  misc_feature 1..516: note = Synthetic
  source 1..516: mol_type = other DNA
cgaccattccagtgtcgaatctgcatgcgcaacttcagccgaagttccaacctgacacggcatatccgcacccacacgggagagaagccttttgcctgcgatatttgtggaaagaagtttgctgacaagcggaccttaatccgccacaccaagattcatactgggtcccagaaaccgttccagtgtaggatatgcatgaggaatttctctcagcggggaaatctagtgcgacatataaggacgcacacaggtgaaaaaccatttgcatgcgacatctgtggcaaaaagtttgcgctgagcttcaacttgactcgtcacacaaaaatccataccggcagtcaaaagccctttcaatgtcgcatttgcatgcgaaacttctcacggagtgacaatcttacgagacatattcgtactcatactggcgagaaacctttcgcttgcgatatatgtggtcgtaagtttgcagaccggagccacttagccaggcacactaagatacacctgcggcagaaggac

SEQ ID NO: 61 - protein, 172 aa, synthetic construct
  REGION 1..172: note = Synthetic
RPFQCRICMRNFSDRSALARHIRTHTGEKPFACDICGKKFARSDNLTRHTKIHTGSQKPFQCRICMRNFSQSGDLTRHIRTHTGEKPFACDICGKKFAVRQTLKQHTKIHTGSQKPFQCRICMRNFSAAGNLTRHIRTHTGEKPFACDICGRKFARSDNLTRHTKIHLRQKD

SEQ ID NO: 62 - DNA, 516 bp, synthetic construct
  misc_feature 1..516: note = Synthetic
  source 1..516: mol_type = other DNA
cgaccattccagtgtcgaatctgcatgcgcaacttcagcgaccggagcgcgctggcacggcatatccgcacccacacgggagagaagccttttgcctgcgatatttgtggaaagaagtttgctcgaagtgacaacttaacgcgccacaccaagattcatactgggtcccagaaaccgttccagtgtaggatatgcatgaggaatttctctcagtcaggggacctcactcgtcatataaggacgcacacaggtgaaaaaccatttgcatgcgacatctgtggcaaaaagtttgcggtacgacagacgcttaaacaacacacaaaaatccataccggcagtcaaaagccctttcaatgtcgcatttgcatgcgaaacttctcagccgctggtaacttgacacgacatattcgtactcatactggcgagaaacctttcgcttgcgatatatgtggtcgtaagtttgcaagatctgataatctaacgcgtcacactaagatacacctgcggcagaaggac

SEQ ID NO: 63 - protein, 28 aa, synthetic construct
  REGION 1..28: note = Synthetic
FQCRICMRNFSQRGNLVRHIRTHTGEKP

SEQ ID NO: 64 - protein, 29 aa, synthetic construct
  REGION 1..29: note = Synthetic
FACDICGKKFALSFNLTRHTKIHTGSQKP

SEQ ID NO: 65 - protein, 28 aa, synthetic construct
  REGION 1..28: note = Synthetic
FQCRICMRNFSRSDNLTRHIRTHTGEKP

SEQ ID NO: 66 - protein, 29 aa, synthetic construct
  REGION 1..29: note = Synthetic
FACDICGKKFADRSHLARHTKIHTGSQKP

SEQ ID NO: 67 - protein, 28 aa, synthetic construct
  REGION 1..28: note = Synthetic
FQCRICMRNFSQKAHLTAHIRTHTGEKP

SEQ ID NO: 68 - protein, 28 aa, synthetic construct
  REGION 1..28: note = Synthetic
FACDICGRKFARSDNLTRHTKIHLRQKD

SEQ ID NO: 69 - protein, 28 aa, synthetic construct
  REGION 1..28: note = Synthetic
FQCRICMRNFSRSSNLTRHIRTHTGEKP

SEQ ID NO: 70 - protein, 29 aa, synthetic construct
  REGION 1..29: note = Synthetic
FACDICGKKFADKRTLIRHTKIHTGSQKP

SEQ ID NO: 71 - protein, 28 aa, synthetic construct
  REGION 1..28: note = Synthetic
FQCRICMRNFSQRGNLVRHIRTHTGEKP

SEQ ID NO: 72 - protein, 29 aa, synthetic construct
  REGION 1..29: note = Synthetic
FACDICGKKFALSFNLTRHTKIHTGSQKP

SEQ ID NO: 73 - protein, 28 aa, synthetic construct
  REGION 1..28: note = Synthetic
FQCRICMRNFSRSDNLTRHIRTHTGEKP

SEQ ID NO: 74 - protein, 28 aa, synthetic construct
  REGION 1..28: note = Synthetic
FACDICGRKFADRSHLARHTKIHLRQKD

SEQ ID NO: 75 - protein, 28 aa, synthetic construct
  REGION 1..28: note = Synthetic
FQCRICMRNFSDRSALARHIRTHTGEKP

SEQ ID NO: 76 - protein, 29 aa, synthetic construct
  REGION 1..29: note = Synthetic
FACDICGKKFARSDNLTRHTKIHTGSQKP

SEQ ID NO: 77 - protein, 28 aa, synthetic construct
  REGION 1..28: note = Synthetic
FQCRICMRNFSQSGDLTRHIRTHTGEKP

SEQ ID NO: 78 - protein, 29 aa, synthetic construct
  REGION 1..29: note = Synthetic
FACDICGKKFAVRQTLKQHTKIHTGSQKP

SEQ ID NO: 79 - protein, 28 aa, synthetic construct
  REGION 1..28: note = Synthetic
FQCRICMRNFSAAGNLTRHIRTHTGEKP

SEQ ID NO: 80 - protein, 28 aa, synthetic construct
  REGION 1..28: note = Synthetic
FACDICGRKFARSDNLTRHTKIHLRQKD

SEQ ID NO: 81 - DNA, 4104 bp, synthetic construct
  misc_feature 1..4104: note = Synthetic
  source 1..4104: mol_type = other DNA
atggacaagaagtactccattgggctcgctatcggtaccaacagcgtcggctgggccgtcattacggacgagtacaaggtgccgagcaaaaaattcaaagttctgggcaataccgatcgccacagcataaagaagaacctcattggagccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagctggtagacagtactgataaggctgacttgcggttgatctatctcgcgctggcgcacatgatcaaatttcggggacacttcctcatcgagggggacctgaacccagacaacagcgatgtcgacaaactctttatccaactggttcagacttacaatcagcttttcgaggagaacccgatcaacgcatccggcgttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagctgcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagctgaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgctcggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagagacgccgatatacaggatgggggcggctgtcaagaaaactgatcaatggcatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaaccggttgtccgactacgacgtggatgctatcgtgccccaaagctttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagctggaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccgcaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatccagcttcgagaaaaaccccatcgactttctcgaagcgaaaggatataaagaggtcaaaaaagacctcatcattaagctgcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 82 - protein, 1368 aa, synthetic construct
  REGION 1..1368: note = Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 83 - DNA, 97 bp, synthetic construct
  misc_feature 1..97: note = Synthetic
  source 1..97: mol_type = other DNA
gaggtaccatagagtgaggcggttttagagctagaaatagcaagttaaaataaggctagtccgttatcaacttgaaaaagtggcaccgagtcggtgc

SEQ ID NO: 84 - RNA, 97 nt, synthetic construct
  misc_feature 1..97: note = Synthetic
  source 1..97: mol_type = other RNA
gaggtaccatagagtgaggcggttttagagctagaaatagcaagttaaaataaggctagtccgttatcaacttgaaaaagtggcaccgagtcggtgc

SEQ ID NO: 85 - DNA, 21 bp, synthetic construct
  misc_feature 1..21: note = Synthetic
  source 1..21: mol_type = other DNA
gaggtaccatagagtgaggcg

SEQ ID NO: 86 - RNA, 21 nt, synthetic construct
  misc_feature 1..21: note = Synthetic
  source 1..21: mol_type = other RNA
gaggtaccatagagtgaggcg

SEQ ID NO: 87 - DNA, 99 bp, synthetic construct
  misc_feature 1..99: note = Synthetic
  source 1..99: mol_type = other DNA
accgaggcgaggatgaagccgaggttttagagctagaaatagcaagttaaaataaggctagtccgttatcaacttgaaaaagtggcaccgagtcggtgc

SEQ ID NO: 88 - RNA, 99 nt, synthetic construct
  misc_feature 1..99: note = Synthetic
  source 1..99: mol_type = other RNA
accgaggcgaggatgaagccgaggttttagagctagaaatagcaagttaaaataaggctagtccgttatcaacttgaaaaagtggcaccgagtcggtgc

SEQ ID NO: 89 - DNA, 23 bp, synthetic construct
  misc_feature 1..23: note = Synthetic
  source 1..23: mol_type = other DNA
accgaggcgaggatgaagccgag

SEQ ID NO: 90 - RNA, 23 nt, synthetic construct
  misc_feature 1..23: note = Synthetic
  source 1..23: mol_type = other RNA
accgaggcgaggatgaagccgag

SEQ ID NO: 91 - DNA, 100 bp, synthetic construct
  misc_feature 1..100: note = Synthetic
  source 1..100: mol_type = other DNA
accgaagccgagaggatactgcaggttttagagctagaaatagcaagttaaaataaggctagtccgttatcaacttgaaaaagtggcaccgagtcggtgc

SEQ ID NO: 92 - RNA, 100 nt, synthetic construct
  misc_feature 1..100: note = Synthetic
  source 1..100: mol_type = other RNA
accgaagccgagaggatactgcaggttttagagctagaaatagcaagttaaaataaggctagtccgttatcaacttgaaaaagtggcaccgagtcggtgc

SEQ ID NO: 93 - DNA, 24 bp, synthetic construct
  misc_feature 1..24: note = Synthetic
  source 1..24: mol_type = other DNA
accgaagccgagaggatactgcag

SEQ ID NO: 94 - RNA, 24 nt, synthetic construct
  misc_feature 1..24: note = Synthetic
  source 1..24: mol_type = other RNA
accgaagccgagaggatactgcag

SEQ ID NO: 95 - DNA, 1569 bp, synthetic construct
  misc_feature 1..1569: note = Synthetic
  source 1..1569: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 96 - protein, 523 aa, synthetic construct
  REGION 1..523: note = Synthetic
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 97 - protein, 1939 aa, synthetic construct
  REGION 1..1939: note = Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGTGGPPKKKRKVAAASRYPRGDALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFPKKKRKVRSKRPAATKKAGQAKKKKLD

SEQ ID NO: 98 - DNA, 112 bp, synthetic construct
  misc_feature 1..112: note = Synthetic
  source 1..112: mol_type = other DNA
gaaacaagctatttgctgatttgtattaggtaccatagagtgaggcgaggatgaagccgagaggatactgcagaggtctctggtgcaatgtgtgtatgtgtgcgtttgtgtg

SEQ ID NO: 99 - DNA, 71 bp, synthetic construct
  misc_feature 1..71: note = Synthetic
  source 1..71: mol_type = other DNA
gaaacaagctatttgctgatttgtattaggtaccatagagtgaggcgaggatgaagccgagaggatactgc

SEQ ID NO: 100 - DNA, 112 bp, synthetic construct; identical to SEQ ID NO: 98
  misc_feature 1..112: note = Synthetic
  source 1..112: mol_type = other DNA

SEQ ID NO: 101 - DNA, 108 bp, synthetic construct
  misc_feature 1..108: note = Synthetic
  misc_difference 52: note = n is a, c, g, or t
  source 1..108: mol_type = other DNA
gaaacaagctatttgctgatttgtattaggtaccatagagtgaggcgaggangaagccgagaggatactgcagaggtctctggtgcaatgtgtgtatgtgtgcgtttg

SEQ ID NO: 102 - DNA, 84 bp, synthetic construct
  misc_feature 1..84: note = Synthetic
  source 1..84: mol_type = other DNA
ttccagtgtcgaatctgcatgcgcaacttcagccagcggggaaacctggtgaggcatatccgcacccacacgggagagaagcct

SEQ ID NO: 103 - DNA, 87 bp, synthetic construct
  misc_feature 1..87: note = Synthetic
  source 1..87: mol_type = other DNA
tttgcctgcgatatttgtggaaagaagtttgctctgagcttcaatctaaccagacacaccaagattcatactgggtcccagaaaccg

SEQ ID NO: 104 - DNA, 85 bp, synthetic construct
  misc_feature 1..85: note = Synthetic
  source 1..85: mol_type = other DNA
ttccagtgtaggatatgcatgaggaatttctctcggagtgacaacttaacgcggcatataaggacgcacacaggtgaaaaaacaa

SEQ ID NO: 105 - DNA, 87 bp, synthetic construct
  misc_feature 1..87: note = Synthetic
  source 1..87: mol_type = other DNA
tttgcatgcgacatctgtggcaaaaagtttgcggaccggtctcaccttgcccgacacacaaaaatccataccggcagtcaaaagccc

SEQ ID NO: 106 - DNA, 84 bp, synthetic construct
  misc_feature 1..84: note = Synthetic
  source 1..84: mol_type = other DNA
tttcaatgtcgcatttgcatgcgaaacttctcacagaaggcccatttgactgcccatattcgtactcatactggcgagaaacct

SEQ ID NO: 107 - DNA, 84 bp, synthetic construct
  misc_feature 1..84: note = Synthetic
  source 1..84: mol_type = other DNA
ttcgcttgcgatatatgtggtcgtaagtttgcacggtcggacaacctcacacgccacactaagatacacctgcggcagaaggac

SEQ ID NO: 108 - DNA, 85 bp, synthetic construct
  misc_feature 1..85: note = Synthetic
  source 1..85: mol_type = other DNA
ttccagtgtcgaatctgcatgcgcaacttcagcccgaatgtccaacctgacacggcatatccgcacccacacgggagagaagcct

SEQ ID NO: 109 - DNA, 87 bp, synthetic construct
  misc_feature 1..87: note = Synthetic
  source 1..87: mol_type = other DNA
tttgcctgcgatatttgtggaaagaagtttgctgacaagcggaccttaatccgccacaccaagattcatactgggtcccagaaaccg

SEQ ID NO: 110 - DNA, 84 bp, synthetic construct
  misc_feature 1..84: note = Synthetic
  source 1..84: mol_type = other DNA
ttccagtgtaggatatgcatgaggaatttctctcagcggggaaatctagtgcgacatataaggacgcacacaggtgaaaaacca

SEQ ID NO: 111 - DNA, 87 bp, synthetic construct
  misc_feature 1..87: note = Synthetic
  source 1..87: mol_type = other DNA
tttgcatgcgacatctgtggcaaaaagtttgcgctgagcttcaacttgactcgtcacacaaaaatccataccggcagtcaaaagccc

SEQ ID NO: 112 - DNA, 84 bp, synthetic construct
  misc_feature 1..84: note = Synthetic
  source 1..84: mol_type = other DNA
tttcaatgtcgcatttgcatgcgaaacttctcacggagtgacaatcttacgagacatattcgtactcatactggcgagaaacct

SEQ ID NO: 113 - DNA, 84 bp, synthetic construct
  misc_feature 1..84: note = Synthetic
  source 1..84: mol_type = other DNA
ttcgcttgcgatatatgtggtcgtaagtttgcagaccggagccacttagccaggcacactaagatacacctgcggcagaaggac

SEQ ID NO: 114 - DNA, 84 bp, synthetic construct
  misc_feature 1..84: note = Synthetic
  source 1..84: mol_type = other DNA
ttccagtgtcgaatctgcatgcgcaacttcagcgaccggagcgcgctggcacggcatatccgcacccacacgggagagaagcct

SEQ ID NO: 115 - DNA, 87 bp, synthetic construct
  misc_feature 1..87: note = Synthetic
  source 1..87: mol_type = other DNA
tttgcctgcgatatttgtggaaagaagtttgctcgaagtgacaacttaacgcgccacaccaagattcatactgggtcccagaaaccg

SEQ ID NO: 116 - DNA, 84 bp, synthetic construct
  misc_feature 1..84: note = Synthetic
  source 1..84: mol_type = other DNA
ttccagtgtaggatatgcatgaggaatttctctcagtcaggggacctcactcgtcatataaggacgcacacaggtgaaaaacca

SEQ ID NO: 117 - DNA, 87 bp, synthetic construct
  misc_feature 1..87: note = Synthetic
  source 1..87: mol_type = other DNA
tttgcatgcgacatctgtggcaaaaagtttgcggtacgacagacgcttaaacaacacacaaaaatccataccggcagtcaaaagccc

SEQ ID NO: 118 - DNA, 84 bp, synthetic construct
  misc_feature 1..84: note = Synthetic
  source 1..84: mol_type = other DNA
tttcaatgtcgcatttgcatgcgaaacttctcagccgctggtaacttgacacgacatattcgtactcatactggcgagaaacct

SEQ ID NO: 119 - DNA, 84 bp, synthetic construct
  misc_feature 1..84: note = Synthetic
  source 1..84: mol_type = other DNA
ttcgcttgcgatatatgtggtcgtaagtttgcaagatctgataatctaacgcgtcacactaagatacacctgcggcagaaggac

SEQ ID NO: 120 - protein, 5 aa, synthetic construct
  REGION 1..5: note = Synthetic Polypeptide
TGEKP

SEQ ID NO: 121 - protein, 6 aa, synthetic construct
  REGION 1..6: note = Synthetic Polypeptide
TGSQKP